(19) United States
(12) Patent Application Publication
(10) Pub. No.: US 2019/0219599 A1
(43) Pub. Date: Jul. 18, 2019

(54) BLOOD-BASED SCREEN FOR DETECTING NEUROLOGICAL DISEASES IN PRIMARY CARE SETTINGS

(71) Applicants: University of North Texas Health Science Center at Fort Worth, Fort Worth, TX (US); Board of Regents, The University of Texas System, Austin, TX (US)

(72) Inventors: Sid E. O'Bryant, Aledo, TX (US); Robert C. Barber, Benbrook, TX (US); Guanghua Xiao, Coppell, TX (US); Dwight German, Dallas, TX (US)

(21) Appl. No.: 16/276,420

(22) Filed: Feb. 14, 2019

Related U.S. Application Data
(63) Continuation-in-part of application No. 14/904,244, filed on Jan. 11, 2016.
(60) Provisional application No. 61/845,121, filed on Jul. 11, 2013.

(51) Int. Cl. G01N 33/68; G06T 7/00

(52) U.S. Cl. CPC G01N 33/6896; G16H 50/70; G01N 2800/2821; G06T 7/0012

(57) ABSTRACT

The present invention includes methods and kits for measuring a level of four or more biomarkers selected from IL1, IL7, TNFα, IL5, IL6, CRP, IL10, TNC, ICAM1, FVII, I309, TNFR1, A2M, TARC, adiponectin, MIP1, eotaxin3, sVCAM1, TPO, FABP, IL18, B2M, SAA, PPY, DJ1, α-synuclein, Ab40, Ab42, tau, alpha-syn, and NfL in a sample separated from a human subject in the primary care setting with neurological disease with a nucleic acid, an immunoassay or an enzymatic activity assay.

### CROSS REFERENCE TO RELATED APPLICATIONS

This application is a continuation-in-part application of U.S. patent application Ser. No. 14/904,244 filed Jan. 11, 2016, which is a national phase application filed under U.S. § 371 of International Application No. PCT/2014/046015, filed on Jul. 9, 2014, which claims the benefit under 35 U.S.C. § 119(e) of U.S. provisional Application No. 61/845,121, filed Jul. 11, 2013. All of which are hereby incorporated by reference in their entirety.

### STATEMENT OF FEDERALLY FUNDED RESEARCH

This invention was made with government support under AG054073, AG051848, AG058252, and AG058537 awarded by The National Institutes of Health. The government has certain rights in the invention.

### FIELD OF INVENTION

The present invention relates in general to the field of screening, detecting and discriminating between neurological diseases within primary care settings, and more particularly, to biomarkers for the detection, screening, and discriminating patients with neurological diseases.

### BACKGROUND OF THE INVENTION

Without limiting the scope of the invention, its background is described in connection with neurological diseases.

The detection and evaluation of disease conditions has progressed greatly as a result of the sequencing of the human genome and the availability of bioinformatics tools. One such system is taught in U.S. Pat. No. 8,430,816, issued to Avinash, et al., for a system and method for analysis of multiple diseases and severities. Briefly, these inventors teach a data processing technique that includes a computer-implemented method for accessing reference deviation maps for a plurality of disease types. The reference deviation maps may include subsets of maps associated with severity levels of respective disease types and a disease severity score may be associated with each severity level. The method is said to also include selecting patient severity levels for multiple disease types based on the subsets of reference deviation maps. Also, the method may include automatically calculating a combined patient disease severity score based at least in part on the disease severity scores associated with the selected patient severity levels, and may include outputting a report based at least in part on the combined patient disease severity score.

Another such invention, is taught in U.S. Pat. No. 8,008,025, issued to Zhang and directed to biomarkers for neurodegenerative disorders. Briefly, this inventor teaches methods for diagnosing neurodegenerative disease, such as Alzheimer's Disease, Parkinson's Disease, and dementia with Lewy body disease by detecting a pattern of gene product expression in a cerebrospinal fluid sample and comparing the pattern of gene product expression from the sample to a library of gene product expression pattern known to be indicative of the presence or absence of a neurodegenerative disease. The methods are also said to provide for monitoring neurodegenerative disease progression and assessing the effects of therapeutic treatment. Also provided are kits, systems and devices for practicing the subject methods.

United States Patent Application Publication No. 2013/0012403, filed by Hu is directed to Compositions and Methods for Identifying Autism Spectrum Disorders. This application is directed to microRNA chips having a plurality of different oligonucleotides with specificity for genes associated with autism spectrum disorders. The invention is said to provide methods of identifying microRNA profiles for neurological and psychiatric conditions including autism spectrum disorders, methods of treating such conditions, and methods of identifying therapeutics for the treatment of such neurological and psychiatric conditions.

Yet another application is United States Patent Application Publication No. 2011/0159527, filed by Schlossmacher, et al., for Methods and Kits for Diagnosing Neurodegenerative Disease. Briefly, the application is said to teach methods and diagnostic kits for determining whether a subject may develop or be diagnosed with a neurodegenerative disease. The method is said to include quantitating the amount of alpha-synuclein and total protein in a cerebrospinal fluid (CSF) sample obtained from the subject and calculating a ratio of alpha-synuclein to total protein content; comparing the ratio of alpha-synuclein to total protein content in the CSF sample with the alpha-synuclein to total protein content ratio in CSF samples obtained from healthy neurodegenerative disease-free subjects; and determining from the comparison whether the subject has a likelihood to develop neurodegenerative disease or making a diagnosis of neurodegenerative disease in a subject. It is said that a difference in the ratio of alpha-synuclein to total protein content indicates that the subject has a likelihood of developing a neurodegenerative disease or has developed a neurodegenerative disease.

### SUMMARY OF THE INVENTION

In one embodiment, the present invention includes a method for detecting biomarkers within a primary care setting comprising: measuring a level of four or more biomarkers selected from IL 1, IL7, TNFα, IL5, IL6, CRP, IL10, TNC, ICAM1, FVII, I309, TNFR1, A2M, TARC, adiponectin, MIP1, eotaxin3, sVCAM1, TPO, FABP, IL18, B2M, SAA, PPY, DJ1, and α-synuclein in a sample separated from a human subject in the primary care setting with neurological disease with a nucleic acid, an immunoassay or an enzymatic activity assay. In one aspect, the neurological disease is selected from the group consisting of Alzheimer's Disease, Parkinson's Disease, Down's syndrome, Frontotemporal dementia, Dementia with Lewy Bodies. In another aspect, the neurological disease is selected from the group consisting of Alzheimer's Disease or Parkinson's Disease. In another aspect, the neurological disease is selected from the group consisting of Alzheimer's Disease or Dementia with Lewy Bodies. In another aspect, the neurological disease is selected from the group consisting of Parkinson's Disease or Dementia with Lewy Bodies. In another aspect, the neurological disease is selected from the group consisting of Alzheimer's Disease, Parkinson's Disease, or Dementia with Lewy Bodies. In another aspect, the method detects 5, 6, 7, 8, 9, 10, 11, 12, or 13 biomarkers of neurological diseases. In another aspect, the sample is serum or plasma. In another aspect, the method further comprises the step of obtaining the following parameters: patient age, and a neurocognitive screening tests, wherein the combination of four or more biomarkers (e.g., serum- or plasma-based, age and the neurocognitive screening tests) are at least 90% accurate in a primary care setting for the determination of Alzheimer's disease when compared to a control subject that does not have a neurological disease or disorder. In another aspect, a profile comprises age, sVCAM1, IL5, B2M, IL6, IL1, adiponexin, Eotaxin, MIP1 and IL10. In another aspect, a profile comprises NFL, PPY, FABP3, IL18, IL7, TARC, TPO, α-syn, Eotaxin3 and IL5, and further comprises Ab40, Ab42, tau, alpha-syn, and NfL. In another aspect, the method further comprises the step of determining one or more of the following parameters: sleep disturbance (yes/no), visual hallucinations (yes/no), psychiatric/personality changes (yes/no), age, neurocognitive screening, and four or more biomarkers for the accurate detection and discrimination between neurodegenerative diseases. In another aspect, the level of expression identified by nucleic acid, an immunoassay or an enzymatic activity assay is selected from fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling. In another aspect, the method is used to screen for at least one of mild AD (CDR global score <=1.0) with an overall accuracy of 94, 95, 96, 97, 98, 99 or 100% (sensitivity (SN), specificity (SP) of (SN=0.94, SP=0.83)), or very early AD (CDR global score =0.5), with an overall accuracy of 91, 92, 93, 94, 95, 96, 97, 98, 99, or 100% (SN=0.97, SP=0.72). In another aspect, the method is used to screen in the primary setting uses a higher specificity than sensitivity, wherein the specificity is in the range of 0.97 to 1.0, and the sensitivity is in the range of 0.80 to 1.0.

In another embodiment, the present invention includes a method for detecting biomarkers in a human patient with neurological disease, the method comprising: detecting a level of four or more proteins selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and α-synuclein by separating the proteins in a sample separated from a human subject in the primary care setting with neurological disease contained in the sample and a molecular marker by electrophoresis; contacting the separated proteins with four or more antibodies that each specifically bind to four or more proteins selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and α-synuclein, and thereafter with a secondary antibody; and then detecting the presence of IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and α-synuclein according to the molecular weight marker. In one aspect, the secondary antibody comprises a fluorescence label, chemiluminescence label, a electrochemiluminescence label, the separation is on a patterned arrayan antibody arrays, a fluorescent resonance transfer label, or a radioactive label. In one aspect, the neurological disease is selected from the group consisting of Alzheimer's Disease, Parkinson's Disease, Down's syndrome, Frontotemporal dementia, Dementia with Lewy Bodies. In another aspect, the neurological disease is selected from the group consisting of Alzheimer's Disease or Parkinson's Disease. In another aspect, the neurological disease is selected from the group consisting of Alzheimer's Disease or Dementia with Lewy Bodies. In another aspect, the neurological disease is selected from the group consisting of Parkinson's Disease or Dementia with Lewy Bodies. In another aspect, the neurological disease is selected from the group consisting of Alzheimer's Disease, Parkinson's Disease, or Dementia with Lewy Bodies. In another aspect, the method detects 5, 6, 7, 8, 9, 10, 11, 12, or 13 biomarkers of neurological diseases. In another aspect, the sample is serum or plasma. In another aspect, the method further comprises the step of obtaining the following parameters: patient age, and a neurocognitive screening tests, wherein the combination of four or more biomarkers, age and the neurocognitive screening tests) are at least 90% accurate in a primary care setting for the determination of Alzheimer's disease when compared to a control subject that does not have a neurological disease or disorder. In another aspect, a profile comprises age, sVCAM1, IL5, B2M, IL6, IL1, adiponexin, Eotaxin, MIP1 and IL10. In another aspect, a profile comprises NFL, PPY, FABP3, IL18, IL7, TARC, TPO, α-syn, Eotaxin3 and IL5, and further comprises Ab40, Ab42, tau, alpha-syn, and NfL. In another aspect, the method further comprises the step of determining one or more of the following parameters: sleep disturbance (yes/no), visual hallucinations (yes/no), psychiatric/personality changes (yes/no), age, neurocognitive screening, and four or more biomarkers for the accurate detection and discrimination between neurodegenerative diseases. In another aspect, the level of expression identified by nucleic acid, an immunoassay or an enzymatic activity assay is selected from fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling. In another aspect, the method is used to screen for at least one of mild AD (CDR global score <=1.0) with an overall accuracy of 94, 95, 96, 97, 98, 99 or 100% (sensitivity (SN), specificity (SP) of (SN=0.94, SP=0.83)), or very early AD (CDR global score=0.5), with an overall accuracy of 91, 92, 93, 94, 95, 96, 97, 98, 99, or 100% (SN=0.97, SP=0.72). In another aspect, the method is used to screen in the primary setting uses a higher specificity than sensitivity, wherein the specificity is in the range of 0.97 to 1.0, and the sensitivity is in the range of 0.80 to 1.0.

In another embodiment, the present invention includes a method of selecting subjects for a clinical trial to evaluate a candidate drug believed to be useful in treating neurological diseases, the method comprising: measuring a level of four or more biomarkers selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and α-synuclein in a sample separated from a human subject in the primary care setting with neurological disease with a nucleic acid, an immunoassay or an enzymatic activity assay; and determining if the subject should participate in the clinical trial based on the results of the identification of the neurodegenerative disease profile of the subject obtained from the step (a), wherein the subject is only selected if the neurodegenerative disease profile if the candidate drug is likely to be useful in treating the neurological disease.

In another embodiment, the present invention includes a method of evaluating the effect of a treatment for a neurological disease, the method comprising: treating a patient for a neurological disease; measuring a level of four or more biomarkers selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and α-synuclein in a sample separated from a human subject in the primary care setting with neurological disease with a nucleic acid, an immunoassay or an enzymatic activity assay; and determining if the treatment reduces the expression of the one or more biomarkers that is statistically significant as compared to any reduction occurring in the second subset of patients that have not been treated or from a prior sample obtained from the patient, wherein a statistically significant reduction indicates that the treatment is useful in treating the neurological disease.

In one embodiment, the present invention includes a method and/or apparatus for screening for neurological disease within a primary care setting comprising: obtaining a blood test sample from a subject in the primary care setting; measuring two or more biomarkers in the blood sample selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and/or α-synuclein; comparing the level of the one or a combination of biomarkers with the level of a corresponding one or combination of biomarkers in a normal blood sample; measuring an increase in the level of the two or more biomarkers in the blood test sample in relation to that of the normal blood sample, which indicates that the subject is likely to have a neurological disease; identifying the neurological disease based on the two biomarkers measured; and selecting a course of treatment for the subject based on the neurological disease predicted. In one aspect, at least one of the biomarker measurements is obtained by a method selected from the group consisting of immunoassay and enzymatic activity assay. In another aspect, the method further comprises advising the individual or a primary health care practitioner of the change in calculated risk. In another aspect, the method further comprises advising the individual or a primary health care practitioner of the change in calculated risk. In another aspect, the method uses 3, 4, 5, 6, 7, 8, 9, 10, 11, 12, or 13 biomarkers to distinguish between neurological diseases. In another aspect, the isolated biological sample is serum or plasma. In another aspect, the sample is a serum sample and upon the initial determination of a neurological disease within the primary care clinic, providing that primary care provider with information regarding the specific type of specialist referral appropriate for that particular blood screen finding and directing the individual to a specialist for that neurological disease and treatment in accordance therewith. In another aspect, the neurological diseases are selected from Alzheimer's Disease, Parkinson's Disease, Down's syndrome, Frontotemporal dementia, Dementia with Lewy Bodies, and neurodegenerative disease. In another aspect, the method further comprises the step of refining the analysis by including the following parameters: patient age, and a neurocognitive screening tests, wherein the combination of two or more serum-based markers, age and the neurocognitive screening tests are at least 90% accurate in a primary care setting for the determination of Alzheimer's disease when compared to a control subject that does not have a neurological disease or disorder. In another aspect, the method further comprises the step of determining one or more of the following parameters: sleep disturbance (yes/no), visual hallucinations (yes/no), psychiatric/personality changes (yes/no), age, neurocognitive screening, and two or more serum-based markers for the accurate detection and discrimination between neurodegenerative diseases. In another aspect, the level of expression of the various proteins is measured by at least one of fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling. In another aspect, the method is used to screen for at least one of mild AD (CDR global score <=1.0) with an overall accuracy of 94, 95, 96, 97, 98, 99 or 100% (sensitivity (SN), specificity (SP) of (SN=0.94, SP=0.83)), or very early AD (CDR global score=0.5), with an overall accuracy of 91, 92, 93, 94, 95, 96, 97, 98, 99, or 100% (SN=0.97, SP=0.72). In another aspect, the method is used to screen in the primary setting used a higher specificity than sensitivity, wherein the specificity is in the range of 0.97 to 1.0, and the sensitivity is in the range of 0.80 to 1.0.

Another embodiment of the present invention includes a method and apparatus for distinguishing between one or more neurological disease states; the method comprising: obtaining from at least one biological sample isolated from an individual suspected of having a neurological disease measurements of biomarkers comprising the biomarkers IL-7 and TNFα; adding the age of the subject and the results from one or more neurocognitive screening tests from the subject (clock drawing, verbal fluency, list learning, sleep disturbances, visual hallucinations, behavioral disturbances, motor disturbances); calculating the individual's risk for developing the neurological disease from the output of a model, wherein the inputs to the model comprise the measurements of the two biomarkers, the subject's age and the results from one or more cognitive tests, and further wherein the model was developed by fitting data from a longitudinal study of a selected population of individuals and the fitted data comprises levels of the biomarkers, the subject's age and the results from one or more cognitive tests and neurological disease in the selected population of individuals; and comparing the calculated risk for the individual to a previously calculated risk obtained from at least one earlier sample from the individual. In one aspect, at least one of the biomarker measurements is obtained by a method selected from at least one of fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling. In another aspect, two or more of the methods for biomarker measurement are used to cross-validate the neurological disease. In another aspect, the method further comprises advising the individual or a health care practitioner of the change in calculated risk. In another aspect, the method further comprises advising the individual or a health care practitioner of the change in calculated risk. In another aspect, the biomarkers further comprise one or more biomarkers selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and/or α-synuclein. In another aspect, the method uses 3, 4, 5, 6, 7, 8, 9, 10, 11, 12, or 13 biomarkers to distinguish the neurological disease. In another aspect, the isolated biological sample is serum or plasma. In another aspect, the sample is a serum sample and upon the initial determination of a neurological disease, directing the individual to a specialist for that neurological disease. In another aspect, the neurological diseases are selected from Alzheimer's Disease, Down's syndrome, Frontotemporal dementia, Dementia with Lewy Bodies, Parkinson's Disease, and dementia. In another aspect, the method is used to exclude one or more neurological diseases selected from Alzheimer's Disease, Down's syndrome, Frontotemporal dementia, Dementia with Lewy Bodies, Parkinson's Disease, and dementia. In another aspect, the method is used to screen in the primary setting used a higher specificity than sensitivity, wherein the specificity is in the range of 0.97 to 1.0, and the sensitivity is in the range of 0.80 to 1.0.

In another embodiment, the present invention also includes a method of performing a clinical trial to evaluate a candidate drug believed to be useful in treating neurological diseases, the method comprising: (a) measuring an two or more biomarkers selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and/or α-synuclein from one or more blood samples obtained from patients suspected of having a neurological disease, the patient's age, and results from one or more neurocognitive screening tests of the patient; (b) administering a candidate drug to a first subset of the patients, and a placebo to a second subset of the patients; (c) repeating step (a) after the administration of the candidate drug or the placebo; and (d) determining if the candidate drug reduces the expression of the one or more biomarkers that is statistically significant as compared to any reduction occurring in the second subset of patients, wherein a statistically significant reduction indicates that the candidate drug is useful in treating the neurological disease. In another aspect, the method further comprises the steps of obtaining one or more additional blood samples from the patient after a predetermined amount of time and comparing the levels of the biomarkers from the one or more additional samples to determine disease progression. In another aspect, the method further comprises the steps of treating the patient for a pre-determined period of time, obtaining one or more additional blood samples from the patient after the predetermined amount of time and comparing the levels of the biomarkers from the one or more additional samples to determine disease progression.

In another embodiment, the present invention also includes a method of selecting subjects for a clinical trial to evaluate a candidate drug believed to be useful in treating neurological diseases, the method comprising: (a) measuring an two or more biomarker selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and/or α-synuclein in a blood samples obtained from the subject, the patient's age and the results from one or more neurocognitive screening tests to determine a neurodegenerative disease profile; and (b) determining if the subject should participate in the clinical trial based on the results of the identification of the neurodegenerative disease profile of the subject obtained from the step (a), wherein the subject is only selected if the neurodegenerative disease profile if the candidate drug is likely to be useful in treating the neurological disease.

In another embodiment, the present invention also includes a method of evaluating the effect of a treatment for a neurological disease, the method comprising: treating a patient for a neurological disease; measuring two or more biomarkers from a blood samples obtained from patients suspected of having a neurological disease, the patient's age, and results from one or more cognitive tests of the patient; and determining if the treatment reduces the expression of the one or more biomarkers that is statistically significant as compared to any reduction occurring in the second subset of patients that have not been treated or from a prior sample obtained from the patient, wherein a statistically significant reduction indicates that the treatment is useful in treating the neurological disease.

In another embodiment, the present invention also includes a method of aiding diagnosis of neurological diseases, comprising: obtaining a blood sample from a human individual; comparing normalized measured levels of IL-7 and TNFα biomarkers from the individual's blood sample to a reference level of each neurological disease diagnosis biomarker; wherein the group of neurological disease diagnosis biomarkers comprises IL-7 and TNFα; and obtaining the patient's age and results from one or more cognitive tests of the patient; wherein the reference level of each neurological disease diagnosis biomarker comprises a normalized measured level of the neurological disease diagnosis biomarker from one or more blood samples of human individuals without neurological disease; and wherein levels of neurological disease diagnosis biomarkers greater than the reference level of each neurological disease diagnosis biomarker, the patient's age and the patient's results from one or more cognitive tests indicate a greater likelihood that the individual suffers from neurological disease. In one aspect, the present invention also includes a method of level of expression of IL-7 and TNF alpha in the blood are elevated when compared to the reference level indicates a greater likelihood that the individual suffers from the neurological disease. In another aspect, the method further comprises the step of determining the blood levels of one or more biomarkers selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and/or α-synuclein. In another aspect, the method uses 3, 4, 5, 6, 7, 8, 9, 10, 11, 12, or 13 biomarkers to distinguish the neurological disease. In another aspect, the levels of CRP and IL10 are lower when compared to the reference level indicates a greater likelihood that the individual suffers from the neurological disease. In another aspect, the method further comprises the steps of obtaining one or more additional blood samples from the patient after a predetermined amount of time and comparing the levels of the biomarkers from the one or more additional samples to determine disease progression. In another aspect, the isolated blood sample is serum sample. In another aspect, the blood sample is a serum sample and upon the initial determination of a neurological disease, directing the individual to a specialist for that neurological disease. In another aspect, the neurological diseases are selected from Alzheimer's Disease, Parkinson's Disease, and dementia. In another aspect, the method is used to screen in the primary setting used a higher specificity than sensitivity, wherein the specificity is in the range of 0.97 to 1.0, and the sensitivity is in the range of 0.80 to 1.0.

In another embodiment, the present invention also includes a rapid-screening kit for aiding diagnosis of a neurological disease in a primary care setting, comprising: one or more reagents for detecting the level of expression of IL-7 and TNFα in a blood sample obtained from a human individual, and one or more neurological screening test sheets; and instructions for comparing normalized measured levels of the IL-7 and TNFα biomarkers from the individual's blood sample to a reference level, the patient's age and the patient's results from the neurological screening tests; wherein the reference level of each neurological disease diagnosis biomarker comprises a normalized measured level of the neurological disease diagnosis biomarker from one or more blood samples of human individuals without neurological disease; and wherein levels of neurological disease diagnosis biomarkers less than the reference level of each neurological disease diagnosis biomarker indicate a greater likelihood that the individual suffers from neurological disease, wherein the test is at least 90% accurate. In another aspect, the level of expression of IL-7 and TNF alpha in the blood are elevated when compared to the reference level indicates a greater likelihood that the individual suffers from the neurological disease. In another aspect, the kit further comprises one or more reagents for detecting the level of expression markers selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and/or α-synuclein. In another aspect, the levels of CRP and IL10 are lower when compared to the reference level indicates a greater likelihood that the individual suffers from the neurological disease. In another aspect, the sample is a serum sample and upon the initial determination of a neurological disease, directing the individual to a specialist for that neurological disease. In another aspect, the neurological diseases are selected from Alzheimer's Disease, Down's syndrome, Frontotemporal dementia, Dementia with Lewy Bodies, Parkinson's Disease, and dementia. In another aspect, the level of expression of the various proteins is measured at least one of the nucleic acid, the protein level, or functionally at the protein level. In another aspect, the level of expression of the various proteins is measured by at least one of fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling.

In another embodiment, the present invention also includes a method of determining one or more neurological disease profiles that best matches a patient profile, comprising: (a) comparing, on a suitably programmed computer, the level of expression of IL-7 and TNFα in a blood sample from a patient suspected of having one or more neurological diseases with reference profiles in a reference database to determine a measure of similarity between the patient profile and each the reference profiles; (b) identifying, on a suitably programmed computer, a reference profile in a reference database that best matches the patient profile based on a maximum similarity among the measures of similarity determined in step (a); and (c) outputting to a user interface device, a computer readable storage medium, or a local or remote computer system; or displaying, the maximum similarity or the disease of the disease cell sample of the reference profile in the reference database that best matches the patient profile. In one aspect, the method further comprises the step of determining the level of expression of one or more markers from a blood sample selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and/or α-synuclein. In another aspect, the method uses 3, 4, 5, 6, 7, 8, 9, 10, 11, 12, or 13 biomarkers to distinguish the neurological disease. In another aspect, the method is used to screen in the primary setting used a higher specificity than sensitivity, wherein the specificity is in the range of 0.97 to 1.0, and the sensitivity is in the range of 0.80 to 1.0.

### BRIEF DESCRIPTION OF THE DRAWINGS

For a more complete understanding of the features and advantages of the present invention, reference is now made to the detailed description of the invention along with the accompanying figures and in which:

FIG. 1 shows data from the Neurodegenerative Panel 1 that assays THPO, FABP3, PPY, IL18, and I309 on an MSD platform from two control participants in duplicate. As can be seen, the assays are highly reliable;

FIG. 2 is a box Plot of Random Forest Risk Scores for AD vs. normal controls (NC);

FIG. 3 is a receiver operation characteristic (ROC) plot of serum biomarker profile;

FIG. 4 is a Gini Plot from Random Forest Biomarker Model;

FIG. 5 is a receiver operation characteristic (ROC) plot of serum biomarker profile; and

FIG. 6 highlights the importance of the relative profiles in distinguishing between neurodegenerative diseases. The relative profiles across disease states varied.

FIG. 7 shows a ROC curve and variable importance plot for Step 1—discriminating Lewy body disease from normal controls.

FIG. 8 shows a ROC curve and variable importance plot.

FIG. 9 shows a ROC Curve and Variable Importance Plot for Proteomic Profile for Detecting Neurodegenerative Disease.

FIG. 10 shows a ROC Curve and Variable Importance Plot for Proteomic Profile for Distinguishing PD from Other Neurodegenerative Diseases

### DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the phrase “primary care clinic”, “primary care setting”, “primary care provider” are used interchangeably to refer to the principal point of contact/consultation for patients within a health care system and coordinates with specialists that the patient may need.

As used herein, the phrase “specialist” refers to a medical practice or practitioner that specializes in a particular disease, such as neurology, psychiatry or even more specifically movement disorders or memory disorders.

As used herein, the following abbreviations are used and can include mammalian version of these genes but in certain embodiments the genes are human genes: IL7—interleukin-7, TNFα—tumor necrosis factor alpha, IL5—interleukin-5, IL6—interleukin-6, CRP—C-reactive protein, IL10—interleukin-10, TNC—Tenascin C, ICAM1—intracellular adhesion molecule 1, FVII—factor VII, I309—chemokine (C-C motif) ligand 1, TNFR1—tumor necrosis factor receptor 1, A2M—alpha-2-microglobulin, TARC—Chemokine (C-C Motif) Ligand 17, eotaxin3, VCAM1—Vascular Cell Adhesion Molecule 1, TPO—thyroid peroxidase, FABP3—fatty acid binding protein 3, IL18—interleukin-18, B2M—beat-2-microglobulin, SAA—serum amyloid A1 cluster, PPY—pancreatic polypeptide, DJ1—Parkinson Protein 7, α-synuclein.

As used herein, the phrase “neurological disease” refers to a disease or disorder of the central nervous system and many include, e.g., neurodegenerative disorders such as AD, Parkinson's disease, mild cognitive impairment (MCI) and dementia and neurological diseases include multiple sclerosis, neuropathies. The present invention will find particular use in detecting AD and for distinguishing the same, as an initial or complete screen, from other neurodegenerative disorders such as Parkinson's Disease, Frontotemporal dementia, Dementia with Lewy Bodies, and Down's syndrome.

As used herein, the terms “Alzheimer's patient”, “AD patient”, and “individual diagnosed with AD” all refer to an individual who has been diagnosed with AD or has been given a probable diagnosis of Alzheimer's Disease (AD).

As used herein, the terms “Parkinson's disease patient”, and “individual diagnosed with Parkinson's disease” all refer to an individual who has been diagnosed with PD or has been given a diagnosis of Parkinson's disease.

As used herein, the terms “Frontotemporal dementia”, and “individual diagnosed with frontotemporal dementia” all refer to an individual who has been diagnosed with FTD or has been given a diagnosis of FTD.

As used herein, the term “Dementia with Lewy bodies” (DLB), and “individual diagnosed with DLB” all refer to an individual who has been diagnosed with DLB or has been given a diagnosis of DLB.

As used herein, the term “Down's syndrome” (DS), and “individual diagnosed with Down's syndrome” all refer to an individual who has been diagnosed with DS or has been given a diagnosis of DS.

As used herein, the phrase “neurological disease biomarker” refers to a biomarker that is a neurological disease diagnosis biomarker.

As used herein, the term “neurological disease biomarker protein”, refers to any of: a protein biomarkers or substances that are functionally at the level of a protein biomarker.

As used herein, methods for “aiding diagnosis” refer to methods that assist in making a clinical determination regarding the presence, or nature, of the neurological disease (e.g., AD, PD, DLB, FTD, DS or MCI), and may or may not be conclusive with respect to the definitive diagnosis. Accordingly, for example, a method of aiding diagnosis of neurological disease can comprise measuring the amount of one or more neurological disease biomarkers in a blood sample from an individual.

As used herein, the term “stratifying” refers to sorting individuals into different classes or strata based on the features of a neurological disease. For example, stratifying a population of individuals with Alzheimer's disease involves assigning the individuals on the basis of the severity of the disease (e.g., mild, moderate, advanced, etc.).

As used herein, the term “predicting” refers to making a finding that an individual has a significantly enhanced probability of developing a certain neurological disease.

As used herein, “biological fluid sample” refers to a wide variety of fluid sample types obtained from an individual and can be used in a diagnostic or monitoring assay. Biological fluid sample include, e.g., blood, cerebral spinal fluid (CSF), urine and other liquid samples of biological origin. Commonly, the samples are treatment with stabilizing reagents, solubilization, or enrichment for certain components, such as proteins or polynucleotides, so long as they do not interfere with the analysis of the markers in the sample.

As used herein, a “blood sample” refers to a biological sample derived from blood, preferably peripheral (or circulating) blood. A blood sample may be, e.g., whole blood, serum or plasma. In certain embodiments, serum is preferred as the source for the biomarkers as the samples are readily available and often obtained for other sampling, is stable, and requires less processing, thus making it ideal for locations with little to refrigeration or electricity, is easily transportable, and is commonly handled by medical support staff.

As used herein, a “normal” individual or a sample from a “normal” individual refers to quantitative data, qualitative data, or both from an individual who has or would be assessed by a physician as not having a disease, e.g., a neurological disease. Often, a “normal” individual is also age-matched within a range of 1, 2, 3, 4, 5, 6, 7, 8, 9 or 10 years with the sample of the individual to be assessed.

As used herein, the term “treatment” refers to the alleviation, amelioration, and/or stabilization of symptoms, as well as delay in progression of symptoms of a particular disorder. For example, “treatment” of AD includes any one or more of: (1) elimination of one or more symptoms of AD, (2) reduction of one or more symptoms of AD, (3) stabilization of the symptoms of AD (e.g., failure to progress to more advanced stages of AD), and (4) delay in onset of one or more symptoms of AD delay in progression (i.e., worsening) of one or more symptoms of AD; and (5) delay in progression (i.e., worsening) of one or more symptoms of AD.

As used herein, the term “fold difference” refers to a numerical representation of the magnitude difference between a measured value and a reference value, e.g., an AD biomarker, a Parkinson's biomarker, a dementia biomarker, or values that allow for the differentiation of one or more of the neurological diseases. Typically, fold difference is calculated mathematically by division of the numeric measured value with the numeric reference value. For example, if a measured value for an AD biomarker is 20 nanograms/milliliter (ng/ml), and the reference value is 10 ng/ml, the fold difference is 2 (20/10=2). Alternatively, if a measured value for an AD biomarker is 10 nanograms/milliliter (ng/ml), and the reference value is 20 ng/ml, the fold difference is 10/20 or −0.50 or −50%).

As used herein, a “reference value” can be an absolute value, a relative value, a value that has an upper and/or lower limit, a range of values; an average value, a median value, a mean value, or a value as compared to a particular control or baseline value. Generally, a reference value is based on an individual sample value, such as for example, a value obtained from a sample from the individual with e.g., a neurological disease such as AD, Parkinson's Disease, or dementia, preferably at an earlier point in time, or a value obtained from a sample from an neurological disease patient other than the individual being tested, or a “normal” individual, that is an individual not diagnosed with AD, Parkinson's Disease, or dementia. The reference value can be based on a large number of samples, such as from AD patients, Parkinson's Disease patients, dementia patients, or normal individuals or based on a pool of samples including or excluding the sample to be tested.

As used herein, the phrase “a predetermined amount of time” is used to describe the length of time between measurements that would yield a statistically significant result, which in the case of disease progression for neurological disease can be 7 days, 2 weeks, one month, 3 months, 6 months, 9 months, 1 year, 1 year 3 months, 1 year 6 months, 1 year 9 months, 2 years, 2 years 3 months, 2 years 6 months, 2 years 9 months, 3, 4, 5, 6, 7, 8, 9 or even 10 years and combinations thereof.

As used herein, the phrases “neurocognitive screening tests”, or “cognitive test” are used to describe one or more tests known to the skilled artisan for measuring cognitive status or impairment and can include but is not limited to: a 4-point clock drawing test, an verbal fluency test, trail making test, list learning test, and the like. The skilled artisan will recognize and know how these tests can be modified, how new tests that measure similar cognitive function can be developed and implemented for use with the present invention.

The differential diagnosis of neurodegenerative diseases is difficult, yet of critical importance for clinical treatment and management as well as for designing therapeutic and prevention trials (1-4). In order for patients to be referred to specialty clinics for advanced assessments and treatment implementation, an appropriate referral is normally required from primary care providers. However, prior work demonstrates that the assessment and management of neurodegenerative diseases is poor in primary care settings5-8 with inappropriate medications frequently administered (9). Given that the average physician visit duration in an ambulatory setting for those age 65+ is approximately 18 minutes (10), primary care providers are in desperate need for a rapid and cost-effective method for screening neurological illness within their geriatric patients so appropriate referrals to a specialist can be made as warranted.

The availability of blood-based screening tools that can be implemented within primary care clinic settings has significant implications. From a clinical standpoint, while fewer than half of physicians surveyed believed screenings for neurodegenerative disease was important, the vast majority of the general public and caregivers believed such screenings were vitally important (11). Additionally, the average physician visit is less than 20 minutes for elderly patients in an ambulatory setting (10), severely limiting the time available for even brief neurological and cognitive assessments. Therefore, primary care providers are in desperate need of a method for determining which patients should be referred to a specialist for advanced clinical evaluation of possible neurodegenerative disease. While a tremendous amount of work has been completed demonstrating the utility of advanced neuroimaging techniques (MRI, fMRI, DTI, PET) in diagnosing neurodegenerative diseases, they are cost prohibitive as the first step in a multi-stage diagnostic process. Due to cost and access, it has been proposed that blood-based biomarkers “will most likely be the prerequisite to future sensitive screening of large populations” at risk for neurodegenerative disease and the baseline in a diagnostic flow approach (12). For example, PET amyloid-beta (Aβ) scans were recently FDA approved for use in the diagnostic process of Alzheimer's disease. If PET Aβ imaging were made available at even $1,000 per exam (less than a third to one tenth of the actual cost) and only 1 million elders were screened annually within primary care settings (there are 40 million Americans age 65+), the cost would be $1 billion (U.S. dollars) annually for neurodegenerative screening. If a blood-based screener were made available at $100/person, the cost would be $100 million annually. If 15% tested positive and went on to PET Aβ imaging ($150 million), the cost savings of this screen—follow-up procedure would be $750 million dollars annually screening less than one fortieth of those who actually need annual screening.

A blood-based tool can easily fit the role as the first step in the multi-stage diagnostic process for neurodegenerative diseases with screen positives being referred to specialist for confirmatory diagnosis and treatment initiation. In fact, this is the process already utilized for the medical fields of cancer, cardiology, infectious disease and many others.

While application of specialty clinic-based screens to primary care settings seems straight forward, this is not the case and no prior procedures will work within primary care settings as demonstrated below. The ability to implement blood-based screenings as the first step in a multi-stage diagnostic process is critical, yet very complicated due to substantially lower base rates of disease presence as compared to specialty clinics13 and this lower base rate has a tremendous impact on the predictive accuracy of test results.

### Example 1. Screening Patients for Neurodegenerative Diseases

Another substantial advancement comes from the current procedure. Specifically, the procedure can also be utilized for screening patients prior to entry into a clinical trial. A major impediment to therapeutic trials aimed at preventing, slowing progression, and/or treating AD is the lack of biomarkers available for detecting the disease14,15. The validation of a blood-based screening tool for AD could significantly reduce the costs of such trials by refining the study entry process. If imaging diagnostics (e.g., Aβ neuroimaging) are required for study entry, only positive screens on the blood test would be referred for the second phase of screening (i.e., PET scan), which would drastically reduce the cost for identification and screening of patients. The new methods for screening of the present invention facilitate recruitment, screening, and/or selection of patients from a broader range of populations and/or clinic settings, thereby offering underserved patient populations the opportunity to engage in clinical trials, which has been a major limitation to the majority of previously conducted trials16.

The present inventors provide for the first time, data that demonstrates the following: a novel procedure can detect and discriminate between neurodegenerative diseases with high accuracy. The current novel procedure which can be utilized for implementation as the first line screen within primary care settings that leads to specific referrals to specialist providers for disease confirmation and initiation of treatment.

Methods. Neurodegenerative disease patients. AD and Control Patients. Non-fasting serum samples from the 300 TARCC participants (150 AD cases, 150 controls) were analyzed. Additionally, 200 plasma samples (100 AD cases and 100 controls), from the same subject group were analyzed. The methodology of the TARCC protocol has been described elsewhere21,22. Briefly, each participant undergoes an annual standardized assessment at one of the five participating TARCC sites that includes a medical evaluation, neuropsychological testing, and a blood draw. Diagnosis of AD is based on NINCDS-ADRDA criteria23 and controls performed within normal limits on psychometric testing. Institutional Review Board approval was obtained at each site and written informed consent is obtained for all participants.

Non-AD Patients. Down's Samples. Serum samples were obtained from 11 male patients diagnosed with Down's syndrome (DS) from the Alzheimer's Disease Cooperative Studies core at the University of California San Diego (UCSD). Parkinson's disease Samples. Serum samples from 49 patients (28 males and 21 females) diagnosed with Parkinson's disease (PD) came from the University of Texas Southwestern Medical Center (UTSW) Movement Disorders Clinic. Dementia with Lewy Bodies (DLB) and Frontotemporal dementia (FTD) Samples. Serum samples from 11 DLB and 19 FTD samples were obtained from the UTSW Alzheimer's Disease Coordinating Center (ADCC).

Serum sample collection. TARCC and UTSW ADC serum samples were collected as follows: (1) non-fasting serum samples was collected in 10 ml tiger-top tubes, (2) allowed to clot for 30 minutes at room temperature in a vertical position, (3) centrifuged for 10 minutes at 1300×g within one hour of collection, (4) 1.0 ml aliquots of serum were transferred into cryovial tubes, (5) Freezerworks™ barcode labels were firmly affixed to each aliquot, and (6) samples placed into −80° C. freezer for storage until use in an assay. Down's syndrome serum samples were centrifuged at 3000 rpm for 10 minutes prior to aliquoting and storage in a −80° C. freezer.

Plasma: (1) non-fasting blood was collected into 10 ml lavender-top tubes and gently invert 10-12 times, (2) centrifuge tubes at 1300×g for 10 minutes within one hour of collection, (3) transfer 1 ml aliquots to cryovial tubes, (4) affix Freezerworks™ barcode labels, and (5) placed in −80° C. freezer for storage.

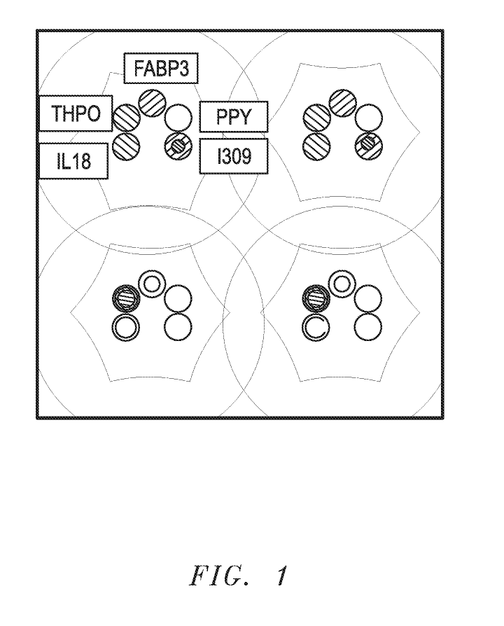

Human serum assays. All samples were assayed in duplicate via a multi-plex biomarker assay platform using electrochemiluminescence (ECL) on the SECTOR Imager 2400A from Meso Scale Discovery (MSD; www.mesoscale.com). The MSD platform has been used extensively to assay biomarkers associated with a range of human disease including AD (24-28). ECL technology uses labels that emit light when electrochemically stimulated, which improves sensitivity of detection of many analytes at very low concentrations. ECL measures have well-established properties of being more sensitive and requiring less volume than conventional ELISAs (26), the gold standard for most assays. The markers assayed were from a previously generated and cross-validated AD algorithm (17,19,29) and included: fatty acid binding protein (FABP3), beta 2 microglobulin, pancreatic polypeptide (PPY), sTNFR1, CRP, VCAM1, thrombopoeitin (THPO), α2 macroglobulin (A2M), exotaxin 3, tumor necrosis factor α, tenascin C, IL-5, IL6, IL7, IL10, IL18, I309, Factor VII, TARC, SAA, and ICAM1, α-synuclein. FIG. 1 illustrates the reliability of the MSD assay of the present invention.

Statistical Analyses. Analyses were performed using R (V 2.10) statistical software (30) and

IBM SPSS19. Chi square and t-tests were used to compare case versus controls for categorical variables (APOE ε4 allele frequency, gender, race, ethnicity, presence of cardiovascular risk factors) and continuous variables (age, education, Mini Mental State Exam [MMSE] and clinical dementia rating sum of boxes scores [CDR-SB]), respectively. The biomarker data was transformed using the Box-Cox transformation. The random forest (RF) prediction model was performed using R package randomForest (V 4.5)(31), with all software default settings. The ROC (receiver operation characteristic) curves were analyzed using R package AUC (area under the curve) was calculated using R package DiagnosisMed (V 0.2.2.2). The sample was randomly divided into training and test samples separately for serum and plasma markers. The RF model was generated in the training set and then applied to the test sample. Logistic regression was used to combine demographic data (i.e. age, gender, education, and APOE4 presence [yes/no]) with the RF risk score as was done in the present inventors' prior work (17,19,29,32). Clinical variables were added to create a more robust diagnostic algorithm given the prior work documenting a link between such variables and cognitive dysfunction in AD (33-36). In order to further refine the algorithm, the biomarker risk score was limited to the smallest set of markers that retained optimal diagnostic accuracy as a follow-up analysis. For the second aim of these studies, support vector machines (SVM) analysis was utilized for multi-classification of all diagnostic groups. A random sample of data from 100 AD cases and controls utilized in the first set of analyses (AD n=51; NC n=49) was selected and combined with serum data from 11 DS, 49 PD, 19 FTD and 11 DLB cases along with 12 additional normal controls (NC) (62 total NCs). The SVM analyses were run on the total combined sample with five-fold cross-validation. SVM is based on the concept of decision planes that define decision boundaries and is primarily a method that performs classification tasks by constructing hyperplanes in a multidimensional space that separates cases of different class labels. An SVM-based method was used with five-fold cross-validation to develop the classifier for the combined samples, and then applied the classifier to predict the combined samples.

Results. As with prior work from the present inventors, the AD patients were significantly older (p<0.001), achieved fewer years of education (p<0.001), scored lower on the MMSE (p<0.001) and higher on the CDR-SB (p<0.001) (see Table 1). There was no significant difference between groups in terms of gender or presence of dyslipidemia, diabetes, or hypertension. The AD group had significantly more APOE4 carriers while the NC group had significantly more individuals who were classified as obese (BMI>=30).

TABLE 1
Demographic Characteristics of Cohort

| | AD | Control | |
|---|---|---|---|
| | (N = 150) | (N = 150) | P-value |
| Gender (male) | 35% | 31% | 0.46 |
| Age (years) | 78.0(8.2) | 70.6(8.9) | <0.001 |
| | 57-94 | 52-90 | |
| Education (years) | 14.0(3.4) | 15.6(2.7) | <0.001 |
| | 0-22 | 10-23 | |
| APOE4 presence (yes/no) | 61% | 26% | <0.001 |
| Hispanic Ethnicity | 5% | 5% | 0.61 |
| Race (non-Hispanic white) | 95% | 97% | 0.49 |
| MMSE | 19.2(6.1) | 29.4(0.9) | <0.001 |
| | 1-30 | 26-30 | |
| CDR-SB | 7.8(44) | 0.0(0.04) | <0.001 |
| | 1-18 | 0-1 | |
| Hypertension (% yes) | 56% | 59% | 0.73 |
| Dyslipidemia (% yes) | 53% | 56% | 0.49 |
| Diabetes (% yes) | 12% | 13% | 0.60 |
| Obese (% yes) | 13% | 24% | 0.04 |

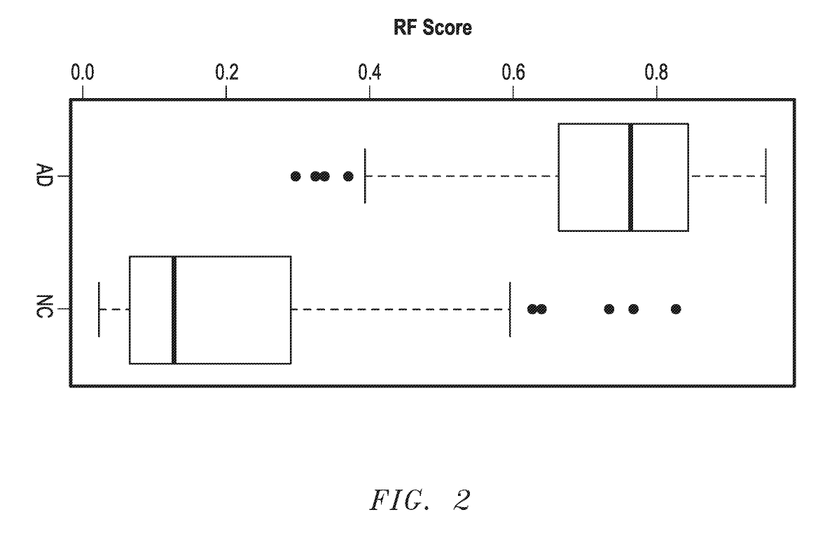

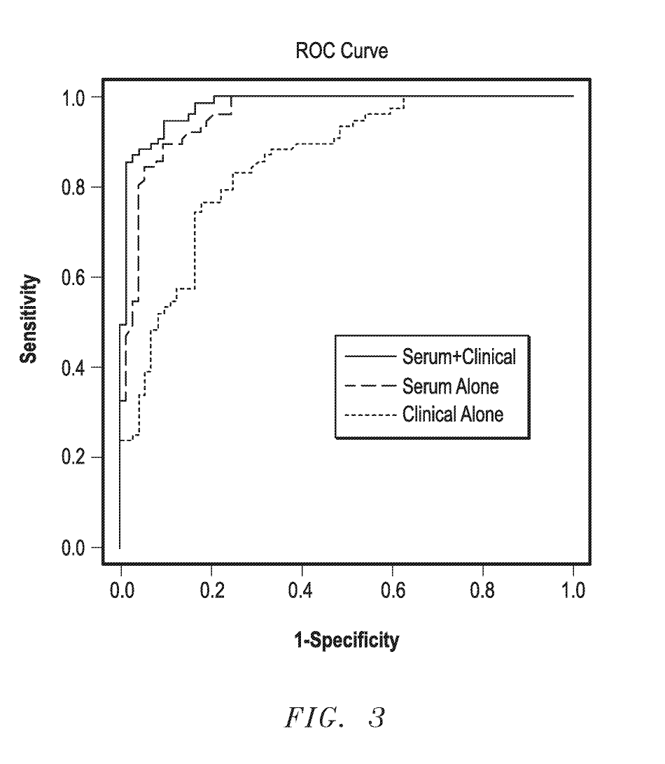

When the serum-based RF biomarker profile from the ECL assays was applied to the test sample, the obtained sensitivity (SN) was 0.90, specificity (SP) was 0.90 and area under the ROC curve (AUC) was 0.96 (See FIGS. 2 and 3, and Table 2).

TABLE 2
Statistical results for AD biomarker sensitivity and specificity and area under the
receiver operating characteristic curve (AUC).

| | AUC | Sensitivity (95% CI) | Specificity (95% CI) |
|---|---|---|---|
| Serum Biomarker alone | 0.96 | 0.90 (0.81, 0.95) | 0.90 (0.82, 0.95) |
| Clinical variables alone | 0.85 | 0.77 (0.66, 0.85) | 0.82 (0.72, 0.89) |
| Biomarkers + Clinical variables | 0.98 | 0.95 (0.87, 0.98) | 0.90 (0.81, 0.95) |
| Abbreviated Biomarker Profile | 0.95 | 0.88 (0.79, 0.94) | 0.92 (0.83, 0.96) |
| (8 proteins) | | | |
| Abbreviated Biomarker Profile | 0.98 | 0.92 (0.84, 0.96) | 0.94 (0.87, 0.98) |
| (8 proteins) + Clinical Variables | | | |
| Plasma Biomaker alone | 0.76 | 0.65 (0.46, 0.74) | 0.790.69, 0.95) |

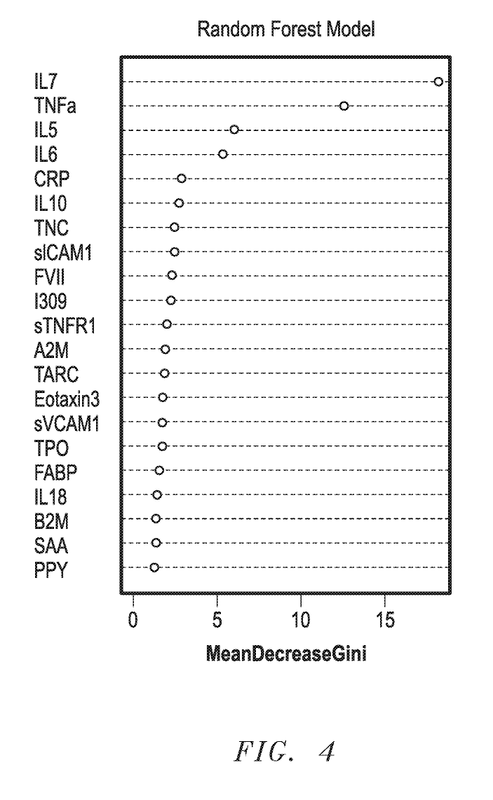

FIG. 3 shows a ROC plot for a serum biomarker profile using 21 serum biomarkers. The plasma-based algorithm yielded much lower accuracy estimates of SN, SP, and AUC of 0.65, 0.79, and 0.76, respectively. Therefore, the remaining analyses focused solely on serum. Inclusion of age, gender, education and APOE4 into the algorithm with the RF biomarker profile increased SN, SP, and AUC to 0.95, 0.90, and 0.98, respectively (Table 2). Next the RF was re-run to determine the optimized algorithm with the smallest number of serum biomarkers. Using only the top 8 markers from the biomarker profile (see FIG. 4) yielded a SN, SP, and AUC of 0.88, 0.92 and 0.95, respectively (see FIG. 5 and Table 2). The addition of age, gender, education and APOE4 genotype increased SN, SP, and AUC to 0.92, 0.94, and 0.98, respectively.

FIG. 4 shows a Gini Plot from Random Forest Biomarker Model demonstrating variable importance and differential expression. FIG. 5 shows a ROC plot using only the top 8 biomarkers for the AD algorithm.

For the SVM multi-classifier analyses to determine if the AD blood-based biomarker profiles could be utilized to discriminate AD from other neurological diseases, analyses were conducted on protein assays from 203 participants (AD n=51, PD n=49, DS n=11, FTD n=19, DLB n=11, NC n=62). Demographic characteristics of this sample are provided in Table 3.

TABLE 3
Demographic characteristics of a second cohort for
multivariate classification

| | AD | PD | DS | FTD | DLB | NC |
|---|---|---|---|---|---|---|
| | N = 51 | N = 49 | N = 11 | N = 19 | N = 11 | N = 61 |
| Age | 78.0 | 68 | 52 | 65.8 | 75.6 | 70 |
| | (9.0) | (9.6) | (2.0) | (8.8) | (4.5) | (9.0) |
| Education | 15.0 | — | — | 14.8 | 14.8 | 16.2 |
| | (3.0) | | | (3.2) | (2.8) | (2.7) |
| Gender | 22 M; | 28 M; | 52 M | 14 M; | 8 M; | 23 M; |
| | 29 F | 21 F | | 5 F | 3F | 38 F |

Note:
information not available regarding education for PD and DS cases.
Abbreviations: AD, Alzheimer's disease.
PD, Parkinson's disease.
DS, Down's syndrome.
FTD, Frontotemporal dementia.
DLB, Lewy Body dementia.
NC, normal controls.

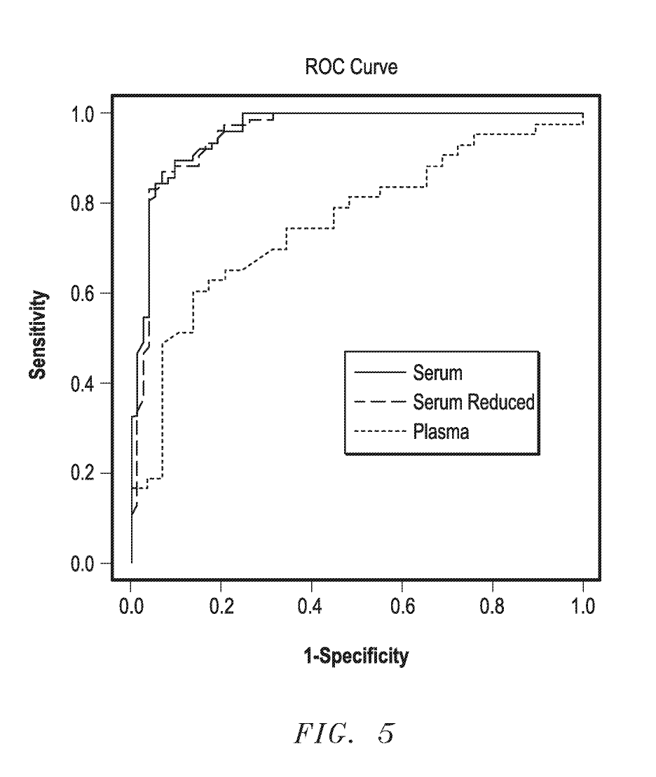

FIG. 6 highlights the importance of the relative profiles in distinguishing between neurodegenerative diseases. The relative profiles across disease states varied. For example, A2M and FVII are disproportionately elevated in DLB and FTD whereas TNFα is disproportionately elevated in AD and lowest in PD and DLB whereas PPY is lowest in PD and highest in DLB. Using the SVM-based algorithm, biomarker profiles combining all proteins were created to simultaneously classify all participants. Surprisingly, the overall accuracy of the SVM was 100% (SN=1.0, SP=1.0) with all of the individuals being correctly classified within their respective categorizations.

Implementing the blood screen in a community-based setting. The 1998 Consensus Report of the Working Group on: “Molecular and Biochemical Markers of Alzheimer's Disease”³⁷ provided guidelines regarding the minimal acceptable performance standards of putative biomarkers for AD. It was stated that sensitivity (SN) and specificity (SP) should be no less than 0.80 with positive predictive value (PPV) of 80% or more, with PPV approaching 90% being best. The report also states that a “high negative predictive value [NPV] would be extremely useful.” The PI and bioinformatics team on this grant have extensive experience calculating diagnostic accuracy statistics, including PPV and NPV^{17-20,38-43}. The important difference between SN/SP and PPV/NPV is that the latter are prediction accuracy statistics (i.e. how correct is a clinician when diagnosing a patient based on the test). PPV/NPV are dependent on base rates of disease presence⁴⁴. With regards to AD, it is estimated that the base rate of disease presence in the community is 11% of those age 65 and above¹³ as compared to 50% or more in specialty clinic settings. PPV and NPV are based on Bayesian statistics and calculated as outlined here:

$$PPV = \frac{( SN \times BR )}{( SN \times BR ) + [ ( 1 - SP ) \times RC ]}$$

$$NPV = \frac{( SP \times RC )}{( SP \times RC ) + [ ( 1 - SN ) \times BR ]}$$

PPV=positive predictive value, SN=sensitivity, BR=base rate, RC=remaining cases, NPV=negative predictive value, SP=specificity. In an 8-protein screen or algorithm, when SP was held at 0.98, SN fell to 0.86. Applying PPV and NPV calculations with an estimated base rate of AD of 11% within the community³, the screen and/or algorithm of the present invention is very accurate and can be used within a community-based setting, that is, at the primary point-of-care. This is in comparison to the minimal requirements to be acceptable based on the 1998 Consensus Report where PPV was less than 35% (see Table 4).

The findings from the present inventors' prior work as well as that from other research groups have also been included for comparison. As is clearly illustrated from above, the current novel procedure is the only procedure that can possibly be utilized in primary care settings in order to have an acceptable accuracy in referrals to specialty clinics. With the exception of the peptoid approach, no other efforts would be better than chance (i.e., 50%) when indicating to a primary care provider that a specialty referral would be needed.

The current approach is 100% at identifying neurodegenerative diseases via the use of overall profiles. Given the very low prevalence of these diseases in the general population, the high accuracy is needed for appropriate referrals to specialist to be made by the primary care practitioners.

Combining specific biomarkers with select cognitive testing. The inventors have demonstrated that molecular profiles could be generated for neuropsychological test performance, and that these profiles accounted for upwards of 50% of the variance in test scores⁴⁸. It was further demonstrated that specific serum-based biomarkers and select cognitive testing can be combined to refine the assessment process and increase diagnostic accuracy. In one example, only the top 2 markers were selected from the serum-algorithm (TNFα and IL7), in conjunction with a single, easy-to-administer cognitive test (in this example a 4-point clock drawing test, but other short and easy tests can be used, e.g., verbal fluency, trail making, list learning, and the like). When these 3 items were combined into a single logistic regression, 92% accuracy was found (SN=0.94, SP=0.90) in distinguishing all AD (n=150) from NC (n=150). When the sample was restricted only to mild AD (CDR global score <=1.0), an overall accuracy of 94% (SN=0.94, SP=0.83) was found. Lastly, and importantly, the sample was restricted only to very early AD (CDR global score=0.5), which resulted in an overall accuracy of 91% (SN=0.97, SP=0.72). These findings clearly demonstrate the possibility of combining specific biomarkers with select cognitive testing to refine the overall algorithm.

In summary, the current approach: (1) is highly accurate at detecting Alzheimer's disease; (2) is highly accurate at detecting and discriminating between neurodegenerative diseases; (3) can be implemented within primary care settings as the first step in a multi-stage diagnostic process; and (4) the combination of specific serum biomarkers and select neurocognitive screening assessments can refine the screening process with excellent accuracy.

Table 6 shows the selection of the specialist for referral, and hence the course of treatment, based on the results of the screen of the two or more biomarkers measured at the primary care center or point of care.

| | Screen Result | | Specialist Referral |
|---|---|---|---|
| Serum Screen in | Alzheimer′s Disease | [symbol] | Memory Disorders Specialist |
| Primary Care Setting | Parkinson′s Disease | [symbol] | Movement Disorders Specialist |
| | Dementia with Lewy Bodies | [symbol] | Specialty Clinic for DLB patients |
| | Frontotemporal Dementia | [symbol] | Specialty Clinic for FTD patients and inclusion of psychiatry |
| | Down′s syndrome | [symbol] | Neurodevelopmental disease specialist and genetic |
| | | | testing/counseling |

### Example 2. Proteomic Signature for Dementia with Lewy Bodies

The inventors sought to determine if a proteomic profile approach developed to detect

Alzheimer's disease would distinguish patients with Lewy body disease from normal controls, and if it would distinguish dementia with Lewy bodies (DLB) from Parkinson's disease (PD).

Stored plasma samples were obtained 145 patients (DLB n=57, PD without dementia n=32, normal controls n=56) enrolled from patients seen in the Behavioral Neurology or Movement Disorders clinics at the Mayo Clinic, Florida. Proteomic assays were conducted and analyzed using the protocols above.

The proteomic profile described herein distinguished the DLB-PD group from controls with a diagnostic accuracy of 0.97, sensitivity of 0.91 and specificity of 0.86. In second step, the proteomic profile distinguished the DLB from PD groups with a diagnostic accuracy of 0.92, sensitivity of 0.94 and specificity of 0.88.

Lewy Body disease is the second most common neurodegenerative disease and clinically may present with dementia as Dementia with Lewy bodies (DLB), or without dementia as Parkinson's disease (PD). DLB was first characterized as a dementia by Kosaka [1] and operationalized diagnostic criteria were initially put forth by McKeith [2] in 1992. Patients who meet consensus criteria for DLB commonly have Lewy-related pathology [3] at autopsy, and in a large dementia autopsy series [4], 25% were found to have Lewy-related pathology. The core clinical features of DLB include parkinsonism, fluctuating cognition, fully formed visual hallucinations and a history of probable REM behavior disorder.[5, 6] [7] There is a subset of patients with Lewy-related pathology who are often not recognized clinically as having DLB [8], in large part because of concomitant Alzheimer (AD) related pathology. Further, the more extensive the tau pathology the harder it is to recognize the DLB phenotype. Multimodality imaging helps to distinguish DLB from AD, but it is an expensive and less viable method for disease detection methods in community samples [9]. Therefore, a front-line, minimally invasive and cost-effective screening method would be of tremendous value to the field.

A major impediment to the development of treatments and clinical trials for neurodegenerative diseases is the lack of sensitive and easily-obtained diagnostic biomarkers [10-14]. The search for biomarkers with diagnostic and prognostic utility in neurodegenerative diseases has grown exponentially, with the majority of work focusing on neuroimaging [15-18] and cerebrospinal fluid (CSF) methodologies [11, 15, 17-19]. Some new promising evidence suggests that CSF may yield a potential biomarker for α-synuclein but replication with a large sample will be needed [20]. While advanced imaging and CSF methods have tremendous potential as confirmatory diagnostic biomarkers of neurodegenerative diseases, accessibility and cost barriers preclude these from being utilized as the first step in this process [12, 13, 21]. Reliable biomarkers of DLB could have many uses, including early and pre-clinical diagnosis, tracking disease progression, and identifying disease endophenotypes [14, 21]. In addition, the advancement of biomarkers may serve to pave the road toward a precision medicine approach to identifying surrogates for therapeutic outcome measures and for the development of disease-modifying treatments [22].

There are no currently validated biomarkers for DLB [23]. It has been proposed that biological markers of the clinical conditions associated with DLB should be “cheap, reliable and reproducible, and make use of biological samples that are easy to obtain”(pg. 1) [13]. Blood-based biomarkers would fulfill these proposed criteria. Additionally, it has been proposed that proteomic biomarker profiling is a promising method for discovering DLB biomarkers [21, 23] because a battery of markers covering a range of biological processes may be required to address the needs of such complex disorders [24]. In fact, profiling analytes associated with multiple disease may highlight novel biological pathways for therapeutic interventions in the dementia syndromes[25]. The inventors' work on blood-based biomarkers of Alzheimer's disease (AD) and PD has consistently shown that a multi-marker approach identifying biomarker profiles of disease presence can yield excellent results [26-28]. The inventors' blood-based biomarker profile provides a cost- and time-effective method for establishing a rapidly scalable multi-tiered neurodiagnostic process [29, 30] for detecting neurodegenerative disease, including DLB. With this initial screening approach, appropriate referrals can be made for subsequent specialty exanimations and confirmatory diagnostic biomarkers (imaging, CSF), following the multi-stage models used for diagnosing cancer [31]. For example, Groveman et al [20] recently demonstrated the accuracy of a rapid and ultra-sensitive seed amplification technique for detection of α-synuclein. In the current proposed context, a blood-based screening tool can be utilized to rule out the vast majority of patients who do not need to undergo lumbar puncture for biomarker confirmatory diagnostics. This approach can also be readily adopted to clinical trials thereby (1) increasing access to broader numbers of patients and (2) significantly reducing screening costs into such novel trials.

In the work described hereinabove, the inventors generated and cross-validated the AD proteomic profile across platforms [26, 32], cohorts [26, 28, 29, 33, 34], species (human, mouse)[32], tissue (brain, serum, plasma)[32] and ethnicities (non-Hispanic white, Mexican American)[26, 35], which is currently being prospectively tested in primary care settings. This same approach was highly accurate in discriminating PD from AD. Here the inventors further shows that the proteomic profile approach to detecting AD [29, 32] is successful in (1) detecting neurodegenerative disease due to synucleinopathy (DLB and PD vs controls) and (2) discriminating amongst neurodegenerative disease due to synucleinopathy (i.e. DLB vs PD). This study was conducted by examination of plasma samples from the Mayo Clinic, Jacksonville. Following the methods described above, the inventors also examined the impact of demographic factors (age, gender, education) on the proteomic profile. Here the inventors utilized the same described above beginning with the discovery phase by using a multi-step approach to determine if this approach can further differentiate neurodegenerative disease and discriminate DLB from PD.

Subjects. The study sample included 145 patients (DLB n=57, PD n=32, normal control n=56) seen though the Alzheimer's Disease Research Center (ADRC) and the Movement Disorders Center at the Mayo Clinic, Florida. All participants underwent a neurologic examination, a Mini-Mental State Examination (MMSE) and diagnosis was based on recent criteria [5, 36]. The DLB patients also underwent, neuropsychological testing, had pathologic confirmation of diffuse or transitional Lewy body disease, and were specifically selected for this study if they had a documented response to cholinesterase inhibitors based the work described above showing that DLB cases who respond to these medications are less likely to have imaging-based AD comorbid pathology [18]. Normal controls were recruited through the ADRC and were all cognitively normal based on neuropsychological testing. All PD-dementia (PDD) cases were not included in this study.

Proteomics. Blood samples were collected per the NACC—Alzheimer's Center guidelines, which also align with the recent guidelines published by an international working group [37]. Briefly, non-fasting sample was collected in an EDTA tube from participants while seated using a 21 g needle, gently inverted 5-10 times and centrifuged at 2000×g for 10 min before being aliquoted into cryovial (polypropylene) tubes and stored at −80° C. All processing was completed within a two-hour timeframe. Samples remained in storage until shipped to the O'Bryant laboratory for assay. Plasma samples were assayed via a multi-plex biomarker assay platform using electrochemiluminescence (ECL) lab using the QuickPlex from Meso Scale Discovery per the inventors' previously published methods using commercially available kits [29, 32]. The MSD platform has been used extensively to assay biomarkers associated with a range of human diseases including AD [38-41]. ECL technology uses labels that emit light when electronically stimulated, which improves the sensitivity of detection of many analytes at very low concentrations. ECL measures have well established properties of being more sensitive and requiring less volume than conventional ELISAs [40], the gold standard for most assays. The inventors recently reported the analytic performance of each of these markers for >1,300 samples across multiple cohorts and diagnoses (normal cognition, MCI, AD) [29]. The assays are reliable and, in the inventors' experience with these assays, again show excellent spiked recovery, dilution linearity, coefficients of variation, as well as detection limits. Inter- and intra-assay variability has been excellent. Internal QC protocols are implemented in addition to manufacturing protocols including assaying consistent controls across batches and assay of pooled standards across lots. To further improve assay performance, assay preparation was automated using a customized Hamilton Robotics StarPlus system. A total of 500 μl of plasma was utilized to assay the following markers (including CV and lowest level of detection) with CVs and LLODs calculated from this automated system using the MSD plates: fatty acid binding protein (CV=2.2 LLOD=13,277 pg/mL), beta 2 microglobulin (CV=7.4, LLOD=32.5 pg/mL), pancreatic polypeptide (CV=4.1, LLOD=390 pg/mL), CRP (CV=2.4; LLOD=2.41 pg/mL), ICAM-1 (CV=4.6; LLOD=1.8 pg/mL), thrombopoeitin (CV=2.2; LLOD=33.1 pg/mL), α2 macroglobulin (CV=2.8; LLOD=5886 pg/mL), exotaxin 3 (CV=18.74 LLOD=3.25 pg/mL), tumor necrosis factor α (CV=3.5; LLOD=0.077 pg/mL), tenascin C (CV=3.7; LLOD=17 pg/mL), interleukin (IL)-5 (CV=12.1; LLOD=0.108 pg/mL), IL6 (CV=4.6; LLOD=0.081 pg/mL), IL7 (CV=12.3; LLOD=0.206 pg/mL), IL10 (CV=6.7; LLOD=0.071 pg/mL), IL18 (CV=3.1; LLOD=6.07 pg/mL), I309 (CV=6.9; LLOD=1.22 pg/mL), Factor VII (CV=2.7; LLOD=49.9 pg/mL), VCAM 1 (CV=2.3; LLOD=6.13 pg/mL), TARC (CV=5.9; LLOD=0.21 pg/mL) SAA (CV=4.4; LLOD=19 pg/mL). As can be seen, analytic performance was excellent with the average CVs across all plates for each analyte being well below standard research use only assays; all CVs<10 and 62% were <5%.

Statistical Analysis. Statistical analyses were conducted using the R (V 3.3.3) statistical software [42], SPSS 24 (IBM) and SAS. Support vector machine (SVM) analyses were conducted to create proteomic profiles specifically for control versus Lewy Body Disease and then DLB vs PD. SVM is based on the concept of decision planes that define decision boundaries and is primarily a classifier method that performs classification tasks by constructing hyperplanes in a multidimensional space that separates cases of different class labels. Diagnostic accuracy was calculated via receiver operating characteristic (ROC) curves. First, SVM analyses were utilized to discriminate controls from Lewy Body Disease (i.e. DLB/PD) with resulting diagnostic accuracy statistics generated (Step 1). Next, SVM analysis was restricted only to those with Lewy Body Disease to discriminate DLB from PD (Step 2) with resulting diagnostic accuracy statistics generated. This two-step process was utilized to allow for the overall algorithm to be more robust and avoid multi-level analyses simultaneously, which reduces risk for error and sample over-identification. Additionally, as described hereinabove, the inventors have demonstrated that the overall profile differs amongst neurodegenerative diseases and, therefore, the multi-step process capitalizes on these overall proteomic profile fluctuations. Lastly, samples from n=53 AD cases were analyzed to provide preliminary analyses on a three-step approach to (1) detect neurodegenerative disease (Alzheimer's disease [AD]/DLB/PD) from controls, (2) discriminate dementia (AD/DLB) from PD and (3) discriminate AD from DLB. These AD cases were also evaluated and clinically diagnosed by the Mayo ADRC. Demographic characteristics of the AD cases are provided in Table 7.

Descriptive statistics of the sample are provided in Table 7. The PD group was younger and included more females than the other two groups. As expected, the DLB group had lower scores on the MMSE.

TABLE 7
Demographic characteristics of the cohort

| | DLB | PD | Normal Control | AD |
|---|---|---|---|---|
| | Mean(SD) | Mean(SD) | Mean(SD) | Mean(SD) |
| N | 57 | 32 | 56 | 53 |
| Age; | 76.03(6.23) | 67.06(11.58) | 76.16(6.07) | 76.12(5.95) |
| mean(sd) | | | | |
| Education | 14.73(3.56) | 15.74(2.49) | 14.47(2.87) | 13.68(3.25) |
| mean(sd) | | | | |
| Gender (% M) | 76.0 | 68.8 | 74.5 | 74.2 |
| MMSE score | 21.13(6.8) | — | 28.04(1.64) | 18.30(5.97) |
| mean(sd) | | | | |

DLB = Dementia with Lewy bodies, PD = Parkinson′s disease, MMSE = Mini Mental State Examination

For the SVM-analyses, a two-step analytic approach was taken. First, the SVM-profile was used to differentiate Lewy Body disease (DLB and PD) from controls. Second, the SVM-analysis was used to differentiate DLB from PD. This two-step approach was utilized as shown above to show that a proteomic profile can be highly accurate in detecting “neurodegenerative disease” in general [29] and therefore, this analyses for discriminating amongst neurodegenerative diseases refines the analysis further without contamination of normal controls in the analytics.

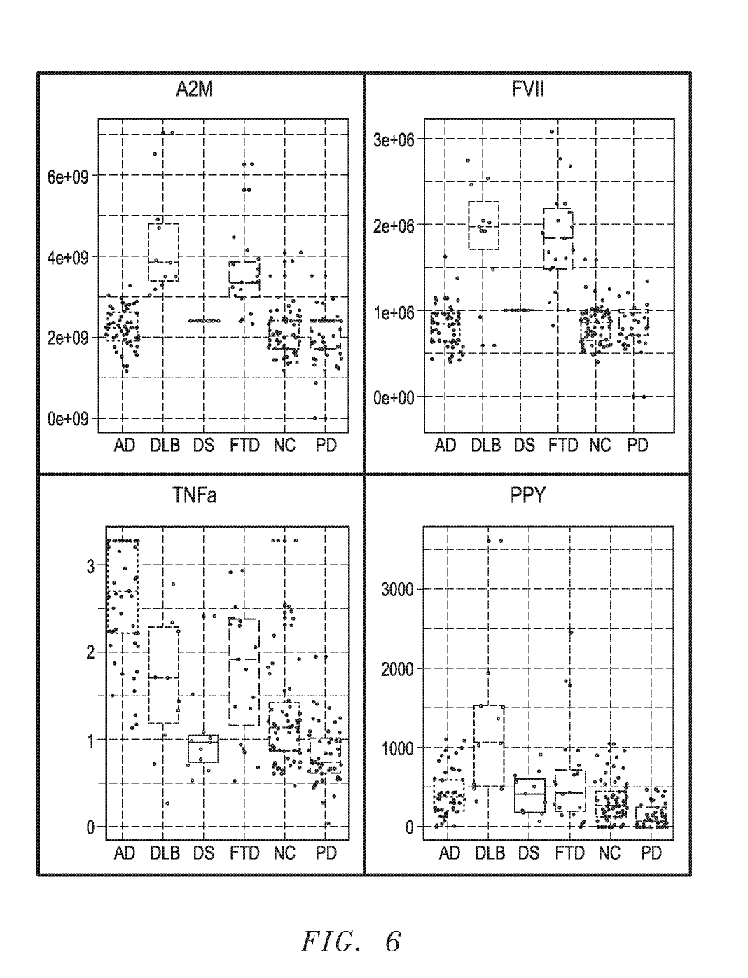

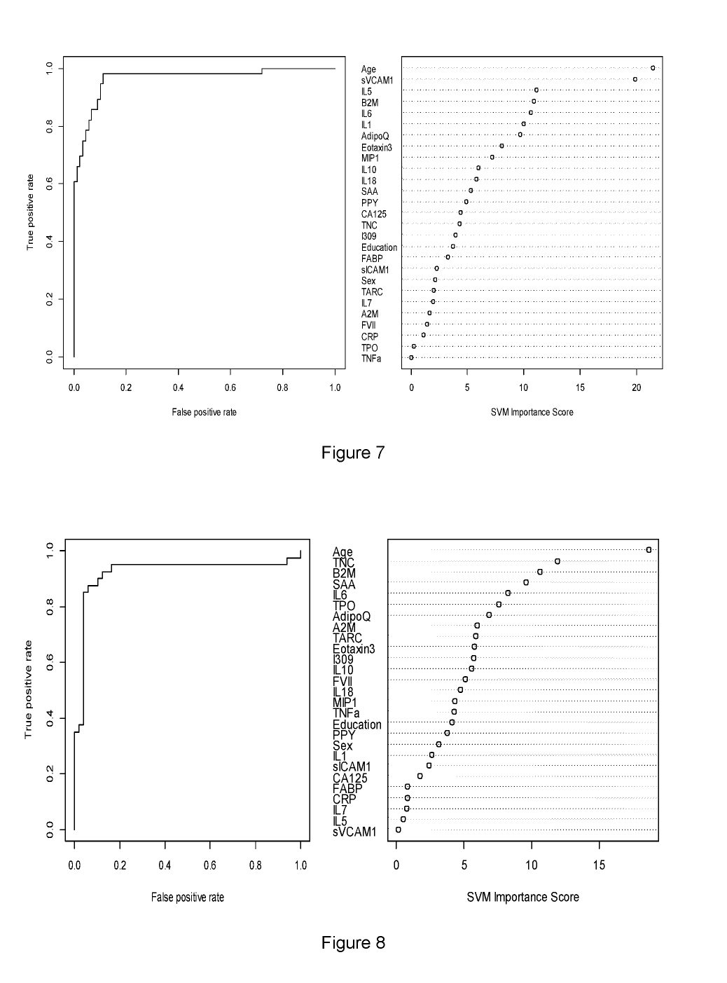

In Step 1, the SVM-based proteomic profile was highly accurate in detecting Lewy Body disease (DLB and PD) as compared to normal controls. The overall AUC of the proteomic profile was 0.94 with a sensitivity (SN) of 0.99 and specificity (SP) of 0.64. As with the inventors' prior work, inclusion of demographic variables (age, gender, education) increased the overall accuracy somewhat with an overall AUC was 0.97 with an decreased SN to 0.91 but increased SP to 0.86. Table 8 shows all of the correct and incorrect predictions while the variable importance plot and ROC curve are presented in FIG. 7.

Table 8: Diagnostic accuracy of blood test in Step 1—discriminating control from Lewy body disease

TABLE 8
Confusion Matrix for SVM-classification for
discriminating Lewy body disease from normal controls

| | SVM Model | |
|---|---|---|
| Predicted | DLB and PD | Normal control |
| LBD | 81 | 8 |
| NC | 8 | 48 |
| Sensitivity | | 91.0% |
| Specificity | | 85.7% |
| AUC | | 0.9653 |

In the Step 2, the overall SVM-proteomic profile also showed good accuracy at distinguishing DLB from PD. In this model, the AUC was 0.84 with SN=0.95 and SN=0.68. Inclusion of demographic variables improved the accuracy to AUC=0.92, SN=0.94 and SP=0.88. Table 9 shows the all classifications (correct and incorrect) while the variable importance plot and ROC curve are presented in FIG. 8.

Next, the inventors conducted preliminary analyses on a three-step algorithmic approach. Here the full algorithm was applied (proteins+demographic variables). In the first step of the model, the inventors sought to detect neurodegenerative disease (AD/DLB/PD) versus controls. With an optimized SVM-risk threshold cut-off of −0.753, the AUC was 0.96 with a SN=0.90 and SP=0.89. In the second step, the inventors sought to discriminate dementia (AD/DLB) from PD which yielded an AUC=0.98, SN=0.96 and SP=0.97. In the third step, the inventors sought to discriminate amongst dementias (DLB vs. AD) and found an AUC=0.96, SN=0.96, SP=0.97.

Table 9 shows the diagnostic accuracy of blood test in Step 2—Discriminating between Dementia with Lewy bodies and Parkinson's disease.

TABLE 9
Confusion Matrix for SVM-classification
for discriminating DLB from PD

| | SVM Model | | |
|---|---|---|---|
| Predicted | DLB | PD | |
| LDB | 46 | 5 | |
| PD | 3 | 35 | |
| Sensitivity | | | 93.9% |
| Specificity | | | 87.5% |
| AUC | | | 0.9204 |

The current example demonstrates, that a multi-step blood-based proteomic profile can accurately distinguish neurodegenerative disease due to synucleinopathy (DLB and PD) from normal controls (AUC=0.97) and DLB from PD (AUC=0.92). Recent work demonstrates that a CSF-based α-synuclein seeding technology can also achieve strong diagnostic accuracy in detecting neurodegenerative disease due to synucleinopathy (93% sensitivity and 100% specificity). While that work requires cross-validation in larger studies, the advancement of the current work in tandem is promising for a sensitive and specific time- and cost-effective multi-step approach for broad-based screening of DLB for prospective studies, clinical trials and routine clinical practice.

While not a limitation of the present invention, by way of explanation, the accuracy of the approach is directly due to the differing overall profiles, which is captured using advanced SVM-analyses. Specifically, as can be seen from FIGS. 7 and 8, the variable importance plots are different in Step 1 versus Step 2. Therefore, by capitalizing on the complexity of the neurodegenerative disease due to synucleinopathy and the number of proteomics available, the inventors can generate bioinformatics profiles. When reviewing the variable importance plots (FIGS. 7 and 8), the overall profiles for discriminating DLB/PD from controls was different than the profile for discriminating DLB from PD. The top 10 markers for discriminating DLB/PD from controls were as follows: age, sVCAM1, IL5, B2M, IL6, IL1, Adipo, Eotaxin, MIP1 and IL10. Not surprisingly the top variable was age in both models. However, the top 2 proteins in this profile were the bottom 2 in the profile for discriminating DLB from PD. In fact, only age, B2M, IL6, adiponectin, and eotaxin overlapped in the top 10 markers in the algorithm (5 out of top 10). Overall, the profile was a mix of inflammatory, metabolic and vascular dysfunction, but at different levels between the categories. The inventors have found that the AD profile is heavily inflammatory in nature as compared to PD and controls. In fact, the AD in adults with Down syndrome is also heavily inflammatory in nature. Therefore, while there are certainly disease-overlapping pathological processes depicted in this work, the profiles are different amongst categories. Prior work has demonstrated that there is a range of biological dysfunction across numerous neurodegenerative diseases. When tau and Aβ are present in DLB, they tend to occur at far less densities than what is typically seen in AD. A recent study showed that in DLB, α-synuclein is a key predictor of disease duration independently, and synergistically with tau and Aβ [Ferman et al., 2018]. It is possible that the proteomic profiles here are picking up on different levels of biological dysfunction due to differing levels of α-synuclein, amyloid and tau pathology. Further work is needed to elucidate the pathological relevance of these overall proteomic profiles.

As shown hereinabove, the inventors have created and validated a proteomic signature for detecting AD across cohorts, species (humans, mice) and tissue (serum, plasma, brain) [26, 28, 29, 32]. Subsequently, the inventors have proposed a multi-tiered neurodiagnostic process for detecting neurodegenerative disease beginning in primary care clinics using blood-based biomarkers [29, 30] which is now being prospectively studied in primary care settings (i.e. Alzheimer's Disease in Primary Care [ADPC] study). The inventors have also demonstrated that the inventors' multi-protein algorithmic approach can discriminate AD from PD [32] as well as controls from “neurodegenerative disease” (i.e. AD, PD, DLB, Down Syndrome) [29]. When compared with AD, the synucleinopathy profile and DLB vs PD profile is different from the AD profile. Additional preliminary analyses were provided here to support the notion that the multi-marker, multi-step profile can also discriminate DLB and PD from AD. Given the sample size, these results are preliminary, but strongly supportive of further work. Therefore, the current work takes a significant step forward in the area of blood-biomarkers for detecting neurodegenerative diseases as it sets the stage for a large-scale, multi-level proteomic-bioinformatic model that takes into account disease-specific profiles across numerous neurodegenerative diseases. The current team is currently assaying large numbers of samples across disease states in order to test this model.

### Example 3. Two-Step Proteomic Signature for Parkinson's Disease

Next, the inventors sought to further validate the proteomic profile approach for detecting Alzheimer's disease would detect Parkinson's disease (PD) and distinguish PD from other neurodegenerative diseases describe hereinabove.

Plasma samples were assayed from 150 patients of the Harvard Biomarkers Study (PD, n=50; other neurodegenerative diseases, n=50; healthy controls n=50) using electrochemiluminescence and Simoa platforms.

The first step proteomic profile distinguished neurodegenerative diseases from controls with a diagnostic accuracy of 0.94. The second step profile distinguished PD cases from other neurodegenerative diseases with a diagnostic accuracy of 0.98. The proteomic profile differed in step 1 versus step 2 suggesting that a multi-step proteomic profile algorithm to detecting and distinguishing between neurodegenerative diseases may be optimal.

This example demonstrates the utility of a multi-tiered blood-based proteomic screening method for detecting individuals with neurodegenerative disease and then distinguishing PD from other neurodegenerative diseases.

Parkinson's disease (PD) is the second most common neurodegenerative disease affecting over 1% of people age 65 and over in the United States [1]. The cost of PD to society was reported to be $23 billion annually in the U.S. in 2005 [2]. Considering the estimated 15% growth in the elderly U.S. population during the last decade, these costs can be expected to increase dramatically as the population ages. Neuropathologically, PD is a progressive disorder of unknown cause affecting multiple neurotransmitter systems. Common non-motor features of the disease include autonomic failure, urinary incontinence, hallucinations, and dementia [3]. While a number of treatments have been developed that improve the “dopaminergic deficit”, no treatment has been demonstrated to slow the neuronal degeneration of the substantia nigra neurons. Novel therapeutic approaches are needed with new disease modifying therapies (DMTs) currently being examined that may ultimately improve patient outcomes.

A major impediment to treatment developments and clinical trials for neurodegenerative diseases is the lack of a sensitive, easily-obtained biomarker of disease presence [4-8]. The “cornerstone” to the development of novel DMTs in PD is the identification and validation of biomarkers of disease presence and progression[9]. Over the last several decades, the search for biomarkers that have diagnostic and prognostic utility in neurodegenerative diseases has grown exponentially[5, 10, 11] with the majority of work focusing on neuroimaging and cerebrospinal (CSF) methods (CSF) [5, 10-14] and increasingly clinical-genetic algorithms [15, 16]. In fact, Aβ PET scanning tracers and CSF assays have been approved by the Food and Drug Administration (FDA) for use in the diagnostic process for Alzheimer's disease (AD) and dopamine transporter single photon emission CT [DaT-SPECT] [17] has been established for PD. Recent work suggests CSF markers may also have utility in the differential diagnosis of neurodegenerative diseases[18]. While advance imaging and CSF methods have tremendous potential as biomarkers of PD and other neurodegenerative diseases, invasiveness, accessibility and cost barriers preclude these from being utilized as initial detection procedures [6, 7, 19, 20]. Therefore, it has been proposed that blood-based methods require additional investigation [21-23] and may serve as first step in a multi-tier detection process [6, 19] similar to the models used in cancer [24].

There has been a surge in the search for blood-based biomarkers for PD[25-27]. Blood-based biomarkers have potential to serve as the initial step in the neurodiagnostic process used in large-scale screening, in primary care settings_[19], as well as screening into novel clinical trials, the latter of which will result in substantial cost savings to the overall trial itself. As is the case with all initial screening tests, the goal of the first-step is to screen out those patients who should not undergo more expensive and invasive confirmatory diagnostic procedures [19]. This is the same model utilized by cancer biomarkers that have received both regulatory and reimbursement approval[24]. The present inventors' work on blood-based biomarkers of Alzheimer's disease (AD) has consistently shown that a multi-marker approach identifying biomarker profiles of disease presence can yield excellent results [28-30]. This blood-based biomarker profile approach provides a cost- and time-effective method for establishing a rapidly scalable multi-tiered neurodiagnostic process [19, 31] for detecting neurodegenerative disease, including PD. With this initial screening approach, appropriate referrals can be made for subsequent specialty exanimations and confirmatory diagnostic biomarkers (imaging, CSF), following the multi-stage models used for diagnosing cancer [24].

This example expands on the validated proteomic profile approach to detecting AD described hereinabove, [31, 32] that is successful in (1) detecting neurodegenerative diseases (PD and other neurodegenerative diseases vs. controls) and (2) discriminating PD from other neurodegenerative disease. This study was conducted by examination of plasma samples from the Harvard Biomarker Study (HBS).

Subjects. The study sample included 150 patients from the Harvard Biomarker Study (HBS; PD n=50; other neurodegenerative diseases n=50, controls n=50). The other neurodegenerative diseases category included AD (n=12), frontotemporal dementia (FTD n=25), progressive supranuclear palsy (n=7), and corticobasal degeneration (n=6) (See Table 10). HBS is a longitudinal, case-control study that tracks clinical phenotypes and linked biospecimens of individuals with neurodegenerative diseases and controls without neurologic disease. High-quality biosamples and high-resolution clinical phenotypes are longitudinally tracked over time. HBS was designed for the primary goal of developing biomarkers that track disease progression and allow go/no go decisions in phase II clinical trials. The HBS specifically fosters research across neurodegenerative diseases, such as the proof-of-concept study described here. HBS has been published extensively [15, 33-40].

TABLE 10
Descriptive Characteristics of the Sample

| | Parkinson's | Neurodegenerative | Healthy |
|---|---|---|---|
| | disease | controls | controls |
| Total N | 50 | 50 | 50 |
| N male/female | 25/25 | 25/25 | 25/25 |
| UPDRS | 49.6 ± 23.9 | — | — |
| Age | 72.4 ± 9.4 | 72.64 ± 10.3 | 69.08 ± 9.7 |
| MMSE | 26.5 ± 3.7 | 20.4 ± 6.7 | 29.2 ± 1.6 |
| PD medications | 36 (72%) | 0 (0%) | 0 (0%) |

Proteomics. Plasma samples were assayed using two technological platforms. The proteomic assays were conducted using two automated systems. The electrochemiluminescence (ECL) assays from the work hereinabove is a previously validated AD blood screen that captured via the multi-plex platform, QuickPlex from Meso Scale Discovery with assay preparation performed via automation using the Hamilton Robotics StarPlus system. The inventors reviewed this analytic performance for each of these markers for >1,300 samples across multiple cohorts and diagnoses (normal cognition, MCI, AD). The results shows that the assays are reliable and the inventors' experience with these assays show excellent spiked recovery, dilution linearity, coefficients of variation, as well as detection limits. Inter- and intra-assay variability has been excellent. A total of 250 μl of plasma was utilized to assay the following markers: fatty acid binding protein, beta2-microglobulin, pancreatic polypeptide, CRP, CAM-1, thrombopoeitin, α2-macroglobulin, exotaxin 3, tumor necrosis factor α, tenascin C, interleukin (IL)-5, IL6, IL7, IL10, IL18, I309, Factor VII, VCAM 1, TARC, SAA. With automation, the average CVs for these assays on >1,000 samples in the inventors' laboratory has been excellent with nearly all having CVs<10% and 62% having CVs<5%. Given the recent surge in the literature examining ultra-sensitive blood-based markers of neuropathological markers in neurodegenerative diseases, here the Simoa assays for Aβ40, Aβ42, tau, α-synuclein and NfL were conducted using the automated HD-1 analyzer from Quanterix. The performance of the assays in the inventors' laboratory from >1,000 samples has been excellent with all CVs<=5%.

Proteomic Profile. As shown hereinabove, the inventors have generated and cross-validated the AD proteomic profile across platforms [28, 32], cohorts [28, 30, 31, 41, 42], species (human, mouse)[32], tissue (brain, serum, plasma)[32] and ethnicities (non-Hispanic white, Mexican American)[28, 43]. A locked-down referent cohort was created for prospective application of the AD Blood Screen[31] and the AD Blood Screen is currently being prospectively studied explicitly as a blood screener for AD in primary care (Alzheimer's Disease in Primary Care [ADPC] study; R01AG058537). In that prior work, the inventors also examined the impact of demographic factors (age, gender, education) on the proteomic profile to ensure that the inventors' proteomic profile performs better than demographics alone and to determine if simple demographic characteristics that are easily obtained can somehow add to the algorithm. Here the inventors' utilized the same approach described above, beginning with the discovery phase. Specifically, the inventors sought to expand on the work described above to determine if the same protein analytes used in the inventors' AD Blood Test algorithm can achieve the same sensitivity and specificity for detecting PD.

Statistical Analysis. Statistical analyses were conducted using R (V 3.3.3) statistical software [44] and SPSS 24 (IBM). Diagnostic accuracy was calculated via receiver operating characteristic (ROC) curves. First, SVM analyses were utilized to discriminate controls from neurodegenerative disease (i.e. PD/Other) with resulting diagnostic accuracy statistics generated (Step 1). Next, SVM analysis was restricted only to PD versus Other neurodegenerative disease (Step 2). SVM analyses were conducted with internal 5-fold cross-validation. In the work described above, the overall proteomic profile varies between different neurodegenerative diseases. Therefore, the two-step approach was used to capitalize on these differences to increase accuracy and also to allow for the overall algorithm to be more robust and avoid multi-level analyses simultaneously. The latter reduces risk for error and sample over-identification.

Descriptive statistics of the sample are provided in Table 10. The average age of the sample was 71.37 (SD=9.9). There were even numbers of males and females across all three groups. An analysis of variance showed there were no significant age differences among the Parkinson's disease group, the healthy control group, and the other neurodegenerative disorders group (F(2,147)=2.04, p=0.13). There were significant group differences in Mini Mental State Exam (MMSE) score among the three groups (F(2,118)=39.9, p=0.001). Tukey's HSD post-hoc analysis revealed that Parkinson's disease participants (M=26.5, SD=3.7) scored significantly lower than healthy controls (M=29.2, SD=1.6), but higher than those with other neurodegenerative diseases (M=20.4, SD=6.7).

In Step 1, the SVM-based proteomic profile was highly accurate in detecting neurodegenerative disease (PD and Other) as compared to normal controls. The overall AUC was 0.94 with an observed sensitivity (SN) of 0.92 and specificity (SP) of 0.65. Table 11 shows all of the correct and incorrect predictions while the variable importance plot and ROC curve are presented in FIG. 1. Inclusion of demographic factors did not significantly change the AUC.

TABLE 11
Accuracy of Step 1 in Detecting Neurodegenerative Diseases

| | SVM Model | |
|---|---|---|
| Predicted | PD/AD/FTD/Others | NC |
| PD/AD/FTD/Others | 92 | 17 |
| NC | 8 | 31 |
| Sensitivity | 92.0% | |
| Specificity | 64.6% | |
| AUC | 0.94 | |

In the Step 2, the overall SVM-proteomic profile also showed excellent accuracy at distinguishing PD from other neurodegenerative diseases. In this model, the AUC was 0.98, SN=0.94 and SP=0.89. Table 3 shows all classifications (correct and incorrect) while the variable importance plot and ROC curve are presented in FIG. 10. Inclusion of demographic factors did not significantly change the AUC.

TABLE 12
Classification Accuracy for Proteomic Profile for Distinguishing
PD from Other Neurodegenerative Diseases

| | SVM Model | |
|---|---|---|
| Predicted | PD | AD/FTD/Others |
| PD | 44 | 7 |
| AD/FTD/Others | 3 | 55 |
| Sensitivity | | 93.6% |
| Specificity | | 88.7% |
| AUC | | 0.98 |

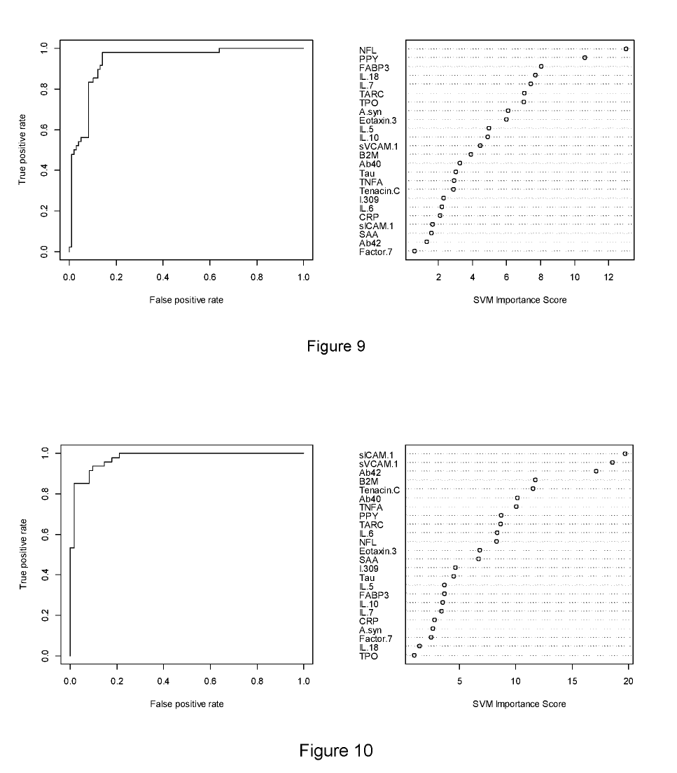

When reviewing the variable importance plots (FIGS. 9 and 10), the overall profiles for discriminating PD/Other neurodegenerative diseases from controls was different than the profile for discriminating PD from Other neurodegenerative diseases as was the case described above. The top 10 markers for discriminating neurodegenerative diseases from controls were as follows: NFL, PPY, FABP3, IL18, IL7, TARC, TPO, α-syn, Eotaxin3 and IL5. However, the top 10 variables for discriminating PD from Other neurodegenerative diseases were ICAM1, VCAM1, Aβ42, B2M, Tenacin C, Aβ40, TNF-α, PPY, TARC, and IL6.

The present work expands on the results shown hereinabove for AD, using; (1) the inventors' AD proteomic algorithm, (2) only the Simoa assays, and (3) all markers combined for discriminating PD from AD as well as PD from controls in this sample. For PD versus AD, the Simoa assays alone yielded an excellent SN of 1.0, but only a SP of only 0.25. The inventors' standard ECL proteomic profile (described hereinabove); however, yielded a superior balance of SN (also 1.0) and SP (0.75). When the Simoa assays were combined with the inventors' standard ECL proteomic panel, there was a modest increase in SP to 0.80. When distinguishing PD from controls, the Simoa assays yielded a SN=0.74 and SP=0.83. The inventors' standard ECL profile yielded an improved SN=0.92 and SP=0.90. The combined algorithm with the inventors' ECL and Simoa assays resulted in an increases SP to 0.94.

The current study further demonstrates that the proteomic profile approach of the present invention can be applied to detecting PD and distinguishing PD from other neurodegenerative diseases. In detecting neurodegenerative disease versus controls, the current AUC was 0.94 with an observed SN of 0.92 and SP of 0.65. When distinguishing PD from other neurodegenerative diseases, the overall accuracy improved to an AUC=0.98, SN=0.94 and SP=0.89.

The identification of some of these markers as of relevance in PD is expected. For example, multiple inflammatory markers such as TNFα and IL6 have previously been linked with PD [45] and inflammation has been shown to improve following exercise interventions in persons with PD_[46, 47]. Mollenhauer et al found FABP to be differentially expressed in PD and dementia with Lewy bodies (DLB) as compared to controls [48] and FABP was among the top 10 markers in discriminating PD from AD in the inventors' prior work (described above). Scherzer and colleagues [40] found differential expression of Parkinson's disease gene α-synuclein (SNCA) in PD and low SNCA transcript abundance predicted cognitive decline longitudinally in PD [40]. Therefore, there is substantial extant literature to support the underlying rationale for these markers being altered PD. However, the prior work never achieved the specificity and sensitivity disclosed herein.

It is important to put these SN and SP estimates into perspective relative to the specific context of use (COU). All first-line screening tools are designed to rule out disease, not rule in disease given the population base rates of disease presence. Therefore, assuming a 20% neurodegenerative disease base rate in the population of those age 65 and above, the SN=0.92 and SP=0.64 would yield a negative predictive power of 0.97 with a positive predictive power of 0.39 using Bayesian statistics for appropriate calculations. This means that a trial would be accurate in saying that a specific patient should not undergo an lumbar puncture, PET scan or additional clinical evaluations 97% of the time, thereby allowing large-scale screening at substantially reduced cost. In Example 1, the inventors' group shows same sorts of calculations for AD clinical trials.

This work also provides novel data when putting the newly designed ultra-sensitive assays of amyloid, tau, α-synuclein and NfL in context with other proteomic markers. In the work taught hereinabove, the algorithm has been highly accurate in detecting both AD and PD. Here further cross-validate the accuracy of the approach for detecting PD in an independent cohort (HBS). In addition, the inventors demonstrate that adding these new markers increases the accuracy. On the other hand, and surprisingly, these new markers were not very accurate at detecting PD or distinguishing PD from AD alone. The SN of 1.0 obtained by both approaches is likely an artifact of sample size and will not hold in larger samples.

Overall, these results demonstrate and further validate the proteomic profiles taught herein. The current team is working towards that goal. In one non-limiting example, the present invention provides clinicians and companies with a rapidly scalable tool (or tools) that can streamline and increase access (while cost containing) to novel clinical trials to improve patient outcomes.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context. In certain embodiments, the present invention may also include methods and compositions in which the transition phrase “consisting essentially of” or “consisting of” may also be used.

As used herein, words of approximation such as, without limitation, “about”, “substantial” or “substantially” refer to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 8, 9, 10, 11, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES—EXAMPLE 1

1. Villemagne V L, Rowe C C. Long night's journey into the day: Amyloid-β imaging in Alzheimer's disease. Journal of Alzheimer's Disease. 2013; 33(SUPPL. 1):S349-S359.

2. Pyykkö O T, Helisalmi S, Koivisto A M, et al. APOE4 predicts amyloid-β in cortical brain biopsy but not idiopathic normal pressure hydrocephalus. Journal of Neurology, Neurosurgery and Psychiatry. 2012; 83(11):1119-1124.

3. Benadiba M, Luurtsema G, Wichert-Ana L, Buchpigel C A, Filho G B. New molecular targets for PET and SPECT imaging in neurodegenerative diseases. Revista Brasileira de Psiquiatria. 2012; 34(SUPPL2):S125-S148.

4. McKeith I G, Fairbairn A F, Bothwell R A, et al. An evaluation of the predictive validity and inter-rater reliability of clinical diagnostic criteria for senile dementia of Lewy body type. Neurology. 1994; 44(5):872-877.

5. Van Den Dungen P, Van Marwijk H W M, Van Der Horst H E, et al. The accuracy of family physicians' dementia diagnoses at different stages of dementia: A systematic review. International Journal of Geriatric Psychiatry. 2012; 27(4):342-354.

6. Löppönen M, Räihä I, Isoaho R, Vahlberg T, Kivelä S-L. Diagnosing cognitive impairment and dementia in primary health care—a more active approach is needed. Age and Ageing. Nov. 1, 2003 2003; 32(6):606-612.

7. Belmin J, Min L, Roth C, Reuben D, Wenger N. Assessment and management of patients with cognitive impairment and dementia in primary care. Journal of Nutrition, Health and Aging. 2012; 16(5):462-467.

8. Maeck L, Haak S, Knoblauch A, Stoppe G. Dementia diagnostics in primary care: a representative 8-year follow-up study in lower saxony, Germany. Dementia & Geriatric Cognitive Disorders. 2008; 25(2):127-134.

9. Fiss T, Thyrian J R, Fendrich K, Van Den Berg N, Hoffmann W. Cognitive impairment in primary ambulatory health care: Pharmacotherapy and the use of potentially inappropriate medicine. International Journal of Geriatric Psychiatry. 2013; 28(2):173-181.

10. Lo A, Ryder K, Shorr R I. Relationship between patient age and duration of physician visit in ambulatory setting: Does one size fit all? Journal of the American Geriatrics Society. 2005; 53(7):1162-1167.

11. Bond J, Graham N, Padovani A, MacKell J, Knox S, Atkinson J. Screening for cognitive impairment, Alzheimer's disease and other dementias: Opinions of European caregivers, payors, physicians and the general public. Journal of Nutrition, Health and Aging. 2010; 14(7):558-562.

12. Schneider P, Hampel H, Buerger K. Biological marker candidates of alzheimer's disease in blood, plasma, and serum. CNS Neuroscience and Therapeutics. 2009; 15(4):358-374.

13. Alzheimer's Association. 2013 Alzheimer's Disease Facts and Figures. Alzheimer's & Dementia. 2013; 9(2):1-72.

14. Shaw L M, Korecka M, Clark C M, Lee V M, Trojanowski J Q. Biomarkers of neurodegeneration for diagnosis and monitoring therapeutics. Nature Reviews. Drug Discovery. 2007; 6(4):295-303.

15. Thal L J, Kantarci K, Reiman E M, et al. The role of biomarkers in clinical trials for Alzheimer disease. Alzheimer Disease & Associated Disorders. 2006; 20(1):6-15.

16. Martin M A, Swider S M, Olinger T, et al. Recruitment of Mexican American adults for an intensive diabetes intervention trial. Ethnicity and Disease. 2011; 21(1):7-12.

17. O'Bryant S E, Xiao G, Barber R, et al. A serum protein-based algorithm for the detection of Alzheimer disease. Archives of Neurology. 2010; 67(9):1077-1081.

18. O'Bryant S, Xiao, G, Barber, R, Reisch, J, Hall, J, Cullum, C M, Doody, R, Fairchild, T, Adams, P, Wilhelmsen, K, & Diaz-Arrastia, R. A blood based algorithm for the detection of Alzheimer's disease. Dementia and Geriatric Cognitive Disorders. 2011; 32:55-62.

19. O'Bryant S E, Xiao G, Barber R, et al. A Blood-Based Screening Tool for Alzheimer's Disease That Spans Serum and Plasma: Findings from TARC and ADNI. PLoS ONE. 2011; 6(12):e28092.

20. O'Bryant S E, Xiao G, Edwards M, et al. Biomarkers of Alzheimer's disease among Mexican Americans. Journal of Alzheimer's Disease. 2013; 34(4):841-849.

21. Waring S, O'Bryant, S E, Reisch, J S, Diaz-Arrastia, R, Knebl, J, Doody, R, for the Texas Alzheimer's Research Consortium. The Texas Alzheimer's Research Consortium longitudinal research cohort: Study design and baseline characteristics. Texas Public Health Journal. 2008; 60(3):9-13.

22. O'Bryant S E, Hobson V, Hall J R, et al. Brain-derived neurotrophic factor levels in alzheimer's disease. Journal of Alzheimer's Disease. 2009; 17(2):337-341.

23. McKhann D, Drockman, D., Folstein, M. et al. Clinical diagnosis of Alzheimer's disease: Report of the NINCDS-ADRDA Work Group. Neurology. 1984; 34:939-944.

24. Bjerke M, Portelius E, Minthon L, et al. Confounding factors influencing amyloid beta concentration in cerebrospinal fluid. International Journal of Alzheimer's Disease. 2010.

25. Kounnas M Z, Danks A M, Cheng S, et al. Modulation of γ-Secretase Reduces β-Amyloid Deposition in a Transgenic Mouse Model of Alzheimer's Disease. Neuron. 2010; 67(5):769-780.

26. Kuhle J, Regeniter A, Leppert D, et al. A highly sensitive electrochemiluminescence immunoassay for the neurofilament heavy chain protein. Journal of Neuroimmunology. 2010; 220(1-2):114-119.

27. Oh E S, Mielke M M, Rosenberg P B, et al. Comparison of conventional ELISA with electrochemiluminescence technology for detection of amyloid-β in plasma. Journal of Alzheimer's Disease. 2010; 21(3):769-773.

28. Alves G, Brønnick K, Aarsland D, et al. CSF amyloid-β and tau proteins, and cognitive performance, in early and untreated Parkinson's Disease: The Norwegian ParkWest study. Journal of Neurology, Neurosurgery and Psychiatry. 2010; 81(10):1080-1086.

29. O'Bryant S E, Xiao G, Barber R, et al. A blood-based algorithm for the detection of Alzheimer's disease. Dementia and Geriatric Cognitive Disorders. 2011; 32(1):55-62.

30. R Development_Core_Team. R: A language and environment for statistical computing. 2009; www.R-project.org.

31. Breiman L. Random forests. Machine Learning. 2001; 45(1):5-32.

32. O'Bryant S E X G, Edwards M, Devous M D, Gupta V, Martins R, Zhang F, Barber R C for the Texas Alzheimer's Research Consortium. Biomarkers of Alzheimer's Disease Among Mexican Americans. Journal of Alzheimer's Disease. 2013, in press.

33. Dickstein D L, Walsh J, Brautigam H, Stockton Jr S D, Gandy S, Hof P R. Role of vascular risk factors and vascular dysfunction in Alzheimer's disease. Mount Sinai Journal of Medicine. 2010; 77(1):82-102.

34. Piazza F, Galimberti G, Conti E, et al. Increased tissue factor pathway inhibitor and homocysteine in Alzheimer's disease. Neurobiology of Aging. 2010.

35. Okereke O I, Selkoe D J, Pollak M N, et al. A profile of impaired insulin degradation in relation to late-life cognitive decline: A preliminary investigation. International Journal of Geriatric Psychiatry. 2009; 24(2):177-182.

36. van Oijen M, Hofman A, Soares H D, Koudstaal P J, Breteler M M. Plasma Abeta (1-40) and Abeta (1-42) and the risk of dementia: a prospective case-cohort study. [see comment].

Lancet Neurology. 2006; 5(8):655-660.

37. Anonymous. Consensus report of the Working Group on: “Molecular and Biochemical Markers of Alzheimer's Disease”. The Ronald and Nancy Reagan Research Institute of the Alzheimer's Association and the National Institute on Aging Working Group. [see comment][erratum appears in Neurobiol Aging 1998 May-June; 19(3):285]. Neurobiology of Aging. 1998; 19(2):109-116.

38. O'Bryant S E, Lucas J A. Estimating the predictive value of the Test of Memory Malingering: An illustrative example for clinicians. Clinical Neuropsychologist. 2006; 20(3):533-540.

39. Bauer L, O'Bryant S E, Lynch J K, McCaffrey R J, Fisher J M. Examining the test of memory malingering trial 1 and word memory test immediate recognition as screening tools for insufficient effort. Assessment. 2007; 14(3):215-222.

40. Clark J H, Hobson V L, O'Bryant S E. Diagnostic accuracy of % retention scores on RBANS verbal memory subtests for the diagnosis of Alzheimer's disease and mild cognitive impairment. Archives of Clinical Neuropsychology. 2010; 25(4):318-326.

41. Duff K, Humphreys Clark J D, O'Bryant S E, Mold J W, Schiffer R B, Sutker P B. Utility of the RBANS in detecting cognitive impairment associated with Alzheimer's disease: Sensitivity, specificity, and positive and negative predictive powers. Archives of Clinical Neuropsychology. 2008; 23(5):603-612.

42. Duff K, Hobson V L, Beglinger L J, O'Bryant S E. Diagnostic accuracy of the RBANS in mild cognitive impairment: Limitations on assessing milder impairments. Archives of Clinical Neuropsychology. 2010; 25(5):429-441.

43. O'Bryant S E, Humphreys J D, Smith G E, et al. Detecting dementia with the mini-mental state examination in highly educated individuals. Archives of Neurology. 2008; 65(7):963-967.

44. O'Bryant S E, Lucas J A, Willis F B, Smith G E, Graff-Radford N R, Ivnik R J. Discrepancies between self-reported years of education and estimated reading level among elderly community-dwelling African-Americans: Analysis of the MOAANS data. Archives of Clinical Neuropsychology. 2007; 22(3):327-332.

45. Doecke J, Laws, S M, Faux, N G, Wilson, W, Burnham, S C, Lam, C P, Mondal, A, Bedo, J, Busy, A I, Brown, B, De Ruyck, K, Ellis, K A, Fowler, C, Gupta, V B, Head, R, Macaulay, L, Pertile, K, Rowe, C C, Rembach, A, Rodrigues, M, Rumble, R, Szoeke, C, Taddei, K, Taddei, T, Trounson, B, Aimes, D, Masters, C L, Martins, R N. Blood-based protein biomarkers for the diagnosis of Alzheimer's disease. Arch Neurol. 2012; published online.

46. Reddy M M, Wilson R, Wilson J, et al. Identification of candidate IgG biomarkers for alzheimer's disease via combinatorial library screening. Cell. 2011; 144(1):132-142.

47. Laske C L T, Stransky E, Hoffmann N, Fallgatter A J, Dietzsch J. Identification of a blood-based biomarker panel for classification of Alzheimer's disease. International Journal of Neuropsychopharmacology. 2011; 14(9):1147-1155.

48. O'Bryant S E G X, Barber R C, Cullum C M, Weiner M, Hall J, Edwards M, Grammas P, Wilhelmsen K, Doody R, Diaz-Arrastia R. Molecular neuropsychology: creation of test-specific blood biomarker algorithms. Dementia and Geriatric Cognitive Disorders. 2013, in press.

### REFERENCES—EXAMPLE 2

1. Kosaka, K., et al., Presenile dementia with Alzheimer-, Pick- and Lewy-body changes. Acta neuropathologica, 1976. 36(3): p. 221-33.

2. McKeith, I. G., et al., Operational criteria for senile dementia of Lewy body type (SDLT). Psychological medicine, 1992. 22(4): p. 911-22.

3. Fujishiro, H., et al., Validation of the neuropathologic criteria of the third consortium for dementia with lewy bodies for prospectively diagnosed cases. Journal of Neuropathology and Experimental Neurology, 2008. 67(7): p. 649-656.

4. Barker, W. W., et al., Relative frequencies of Alzheimer disease, Lewy body, vascular and frontotemporal dementia, and hippocampal sclerosis in the State of Florida Brain Bank. Alzheimer Dis Assoc Disord, 2002. 16(4): p. 203-12.

5. McKeith, I. G., et al., Diagnosis and management of dementia with Lewy bodies: Fourth consensus report of the DLB Consortium. Neurology, 2017. 89(1): p. 88-100.

6. Ferman, T. J., et al., Inclusion of RBD improves the diagnostic classification of dementia with Lewy bodies. Neurology., 2011. 77(9): p. 875-882.

7. Murray, M. E., et al., MRI and pathology of REM sleep behavior disorder in dementia with Lewy bodies. Neurology, 2013. 81(19): p. 1681-9.

8. Halliday, G. M., et al., Neuropathology underlying clinical variability in patients with synucleinopathies. Acta neuropathologica, 2011. 122(2): p. 187-204.

9. Kantarci, K., et al., Multimodality imaging characteristics of dementia with Lewy bodies. Neurobiology of aging, 2012. 33(9): p. 2091-105.

10. Henriksen K, O. B. S., Hampel H, Trojanowski J Q, Montine T J, Jeromin A et al, The future of blood-based biomarkers for Alzheimer's disease. Alzheimer's & Dementia, in press, 2013.

11. Thal, L. J., et al., The role of biomarkers in clinical trials for Alzheimer disease. Alzheimer Disease & Associated Disorders, 2006. 20(1): p. 6-15.

12. Schneider, P., H. Hampel, and K. Buerger, Biological marker candidates of alzheimer's disease in blood, plasma, and serum. CNS Neuroscience and Therapeutics, 2009. 15(4): p. 358-374.

13. Eller, M. and D. R. Williams, α-Synuclein in Parkinson disease and other neurodegenerative disorders. Clinical Chemistry and Laboratory Medicine, 2011. 49(3): p. 403-408.

14. Sinha, N., M. Firbank, and J. T. O'Brien, Biomarkers in dementia with Lewy bodies: A review. International Journal of Geriatric Psychiatry, 2012. 27(5): p. 443-453.

15. Colloby, S. J., et al., A comparison of 99mTc-exametazime and 123I-FP-CIT SPECT imaging in the differential diagnosis of Alzheimer's disease and dementia with Lewy bodies. International Psychogeriatrics, 2008. 20(6): p. 1124-1140.

16. Higuchi, M., et al., Glucose hypometabolism and neuropathological correlates in brains of dementia with Lewy bodies. Experimental Neurology, 2000. 162(2): p. 247-256.

17. McKeith, I., et al., Sensitivity and specificity of dopamine transporter imaging with 123I-FP-CIT SPECT in dementia with Lewy bodies: a phase III, multicentre study. Lancet Neurology, 2007. 6(4): p. 305-313.

18. Graff-Radford, J., et al., Imaging and acetylcholinesterase inhibitor response in dementia with Lewy bodies. Brain, 2012. 135(8): p. 2470-2477.

19. Anonymous, Consensus report of the Working Group on: “Molecular and Biochemical Markers of Alzheimer's Disease”. The Ronald and Nancy Reagan Research Institute of the Alzheimer's Association and the National Institute on Aging Working Group. [see comment][erratum appears in Neurobiol Aging 1998 May-June; 19(3):285]. Neurobiology of Aging, 1998. 19(2): p. 109-16.

20. Groveman, B. R., et al., Rapid and ultra-sensitive quantitation of disease-associated alphα-synuclein seeds in brain and cerebrospinal fluid by alphaSyn RT-QuIC. Acta Neuropathol Commun, 2018. 6(1): p. 7.

21. Henchcliffe, C., R. Dodel, and M. F. Beal, Biomarkers of Parkinson's disease and Dementia with Lewy bodies. Progress in Neurobiology, 2011. 95(4): p. 601-613.

22. Hampel H, O. B. S., Castrillo J I, Ritchie C, Rojkova K, Benda N, Nistico R, Frank R A, Dubois B, Escott-Price V, Lista S, Precision Medicine: The Golden Gate for detection, treatment and prevention of Alzheimer's disease. Journal of Prevention of Alzheimer's Disease, 2016 3(4): p. 243-259.

23. Ho, G. J., et al., Bridging molecular genetics and biomarkers in Lewy body and related disorders. International Journal of Alzheimer's Disease, 2011.

24. Shtilbans, A. and C. Henchcliffe, Biomarkers in Parkinson's disease: An update. Current Opinion in Neurology, 2012. 25(4): p. 460-465.

25. Hu, W. T., et al., Biomarker discovery for Alzheimer's disease, frontotemporal lobar degeneration, and Parkinson's disease. Acta Neuropathologica, 2010. 120(3): p. 385-399.

26. O'Bryant, S. E., et al., A serum protein-based algorithm for the detection of Alzheimer disease. Archives of Neurology, 2010. 67(9): p. 1077-1081.

27. O'Bryant, S. E., et al., A blood-based algorithm for the detection of Alzheimer's disease. Dementia and Geriatric Cognitive Disorders, 2011. 32(1): p. 55-62.

28. O'Bryant, S. E., et al., A Blood-Based Screening Tool for Alzheimer's Disease That Spans Serum and Plasma: Findings from TARC and ADNI. PLoS ONE, 2011. 6(12): p. e28092.

29. O'Bryant S E, E. M., Johnson L A, Hall J A, Villarreal A E, Britton G B, Quiceno M, Cullum C M, Graff-Radford N R, A Blood Screening Test for Alzheimer's Disease. Alzheimer's & Dementia: Diagnosis, Assessment & Disease Monitoring, 2016. 3: p. 83-90.

30. O'Bryant, S. E., et al., Blood-based biomarkers in Alzheimer disease: Current state of the science and a novel collaborative paradigm for advancing from discovery to clinic. Alzheimers Dement, 2017. 13(1): p. 45-58.

31. Gold, L. S., et al., The emergence of diagnostic imaging technologies in breast cancer: Discovery, regulatory approval, reimbursement, and adoption in clinical guidelines. Cancer Imaging, 2012. 12(1): p. 13-24.

32. O'Bryant, S. E., et al., Validation of a serum screen for alzheimer's disease across assay platforms, species, and tissues. Journal of Alzheimer's Disease, 2014. 42(4): p. 1325-1335.

33. Villarreal A E, O. B. S., Edwards M, Grajales S & Britton G B for the Panama Aging Research Initiative, Serum-based protein profiles of Alzheimer's disease and mild cognitive impairment in elderly Hispanics. Neurodegener Dis Manag, 2016, in press.

34. Edwards, M., et al., Molecular markers of amnestic mild cognitive impairment among Mexican Americans. J Alzheimers Dis, 2016. 49(1): p. 221-8.

35. O'Bryant, S. E., et al., Biomarkers of Alzheimer's disease among Mexican Americans. Journal of Alzheimer's Disease, 2013. 34(4): p. 841-849.

36. Postuma, R. B., et al., MDS clinical diagnostic criteria for Parkinson's disease. Mov Disord, 2015. 30(12): p. 1591-601.

37. O'Bryant S E, G. V., Henriksen K, Edwards M, Jeromin A, Lista S, Bazenet C, Soares H, Lovestone S, Hampel H, Montine T, Blennow K, Foroud T, Carrillo M, Graff-Radford N, Laske C, Breteler M, Shaw L, Trojanowski J Q, Schupf N, Rissman R, Fagan A M, Oberoi P, Umek R, Weiner M W, Grammas P, Posner H & Martins R, Guidelines for the standardization of preanalytic variables for blood-based biomarker studies in Alzheimer's disease. Alzheimer's & Dementia, 2015. 11: p. 549-60.

38. Alves, G., et al., CSF amyloid-β and tau proteins, and cognitive performance, in early and untreated Parkinson's Disease: The Norwegian ParkWest study. Journal of Neurology, Neurosurgery and Psychiatry, 2010. 81(10): p. 1080-1086.

39. Bjerke, M., et al., Confounding factors influencing amyloid beta concentration in cerebrospinal fluid. International Journal of Alzheimer's Disease, 2010.

40. Kuhle, J., et al., A highly sensitive electrochemiluminescence immunoassay for the neurofilament heavy chain protein. Journal of Neuroimmunology, 2010. 220(1-2): p. 114-119.

41. Oh, E. S., et al., Comparison of conventional ELISA with electrochemiluminescence technology for detection of amyloid-βin plasma. Journal of Alzheimer's Disease, 2010. 21(3): p. 769-773.

42. R_Development_Core_Team. R: A language and environment for statistical computing. 2009; Available from: http://www.R-project.org.

43. Nakamura, A., et al., High performance plasma amyloid-beta biomarkers for Alzheimer's disease. Nature, 2018. 554(7691): p. 249-254.

44. Wildburger, N. C., et al., Amyloid-beta Plaques in Clinical Alzheimer's Disease Brain Incorporate Stable Isotope Tracer In Vivo and Exhibit Nanoscale Heterogeneity. Front Neurol, 2018. 9: p. 169.

### REFERENCES—EXAMPLE 3

1. Wright Willis, A., et al., Geographic and ethnic variation in Parkinson disease: a population-based study of US Medicare beneficiaries. Neuroepidemiology, 2010. 34(3): p. 143-51.

2. Huse, D. M., et al., Burden of illness in Parkinson's disease. Mov Disord, 2005. 20(11): p. 1449-54.

3. Hely, M. A., et al., The Sydney multicenter study of Parkinson's disease: the inevitability of dementia at 20 years. Mov Disord, 2008. 23(6): p. 837-44.

4. Henriksen K, O. B. S., Hampel H, Trojanowski J Q, Montine T J, Jeromin A et al, The future of blood-based biomarkers for Alzheimer's disease. Alzheimer's & Dementia, in press, 2013.

5. Thal, L. J., et al., The role of biomarkers in clinical trials for Alzheimer disease. Alzheimer Disease & Associated Disorders, 2006. 20(1): p. 6-15.

6. Schneider, P., H. Hampel, and K. Buerger, Biological marker candidates of alzheimer's disease in blood, plasma, and serum. CNS Neuroscience and Therapeutics, 2009. 15(4): p. 358-374.

7. Eller, M. and D. R. Williams, α-Synuclein in Parkinson disease and other neurodegenerative disorders. Clinical Chemistry and Laboratory Medicine, 2011. 49(3): p. 403-408.

8. Sinha, N., M. Firbank, and J. T. O'Brien, Biomarkers in dementia with Lewy bodies: A review. International Journal of Geriatric Psychiatry, 2012. 27(5): p. 443-453.

9. Gerlach, M., et al., Biomarker candidates of neurodegeneration in Parkinson's disease for the evaluation of disease-modifying therapeutics. Journal of Neural Transmission, 2012. 119(1): p. 39-52.

10. Anonymous, Consensus report of the Working Group on: “Molecular and Biochemical Markers of Alzheimer's Disease”. The Ronald and Nancy Reagan Research Institute of the Alzheimer's Association and the National Institute on Aging Working Group. [see comment][erratum appears in Neurobiol Aging 1998 May-June; 19(3):285]. Neurobiology of Aging, 1998. 19(2): p. 109-16.

11. O'Bryant, S. E., et al., Brain-derived neurotrophic factor levels in alzheimer's disease. Journal of Alzheimer's Disease, 2009. 17(2): p. 337-341.

12. Graff-Radford, J., et al., Imaging and acetylcholinesterase inhibitor response in dementia with Lewy bodies. Brain, 2012. 135(8): p. 2470-2477.

13. Colloby, S. J., et al., A comparison of 99mTc-exametazime and 123I-FP-CIT SPECT imaging in the differential diagnosis of Alzheimer's disease and dementia with Lewy bodies. International Psychogeriatrics, 2008. 20(6): p. 1124-1140.

14. McKeith, I., et al., Sensitivity and specificity of dopamine transporter imaging with 123I-FP-CIT SPECT in dementia with Lewy bodies: a phase III, multicentre study. Lancet Neurology, 2007. 6(4): p. 305-313.

15. Liu, G., et al., Specifically neuropathic Gaucher's mutations accelerate cognitive decline in Parkinson's. Ann Neurol, 2016. 80(5): p. 674-685.

16. Liu, G., et al., Prediction of cognition in Parkinson's disease with a clinical-genetic score: a longitudinal analysis of nine cohorts. Lancet Neurol, 2017. 16(8): p. 620-629.

17. Park, E., et al., Differential Diagnosis of Patients with Inconclusive Parkinsonian Features Using [18F]FP-CIT PET/CT. Nuclear Medicine and Molecular Imaging, 2014. 48(2): p. 106-113.

18. Kaerst, L., et al., Using cerebrospinal fluid marker profiles in clinical diagnosis of dementia with lewy bodies, Parkinson's disease, and Alzheimer's disease. Journal of Alzheimer's Disease, 2014. 38(1): p. 63-73.

19. O'Bryant, S. E., et al., Blood-based biomarkers in Alzheimer disease: Current state of the science and a novel collaborative paradigm for advancing from discovery to clinic. Alzheimers Dement, 2017. 13(1): p. 45-58.

20. Henchcliffe, C., R. Dodel, and M. F. Beal, Biomarkers of Parkinson's disease and Dementia with Lewy bodies. Progress in Neurobiology, 2011. 95(4): p. 601-613.

21. Shtilbans, A. and C. Henchcliffe, Biomarkers in Parkinson's disease: An update. Current Opinion in Neurology, 2012. 25(4): p. 460-465.

22. Henriksen, K., et al., The future of blood-based biomarkers for Alzheimer's disease. Alzheimer's and Dementia, 2014. 10(1): p. 115-131.

23. Hennecke, G. and C. R. Scherzer, RNA biomarkers of Parkinson's disease: developing tools for novel therapies. Biomark Med, 2008. 2(1): p. 41-53.

24. Gold, L. S., et al., The emergence of diagnostic imaging technologies in breast cancer: Discovery, regulatory approval, reimbursement, and adoption in clinical guidelines. Cancer Imaging, 2012. 12(1): p. 13-24.

25. Hansson, O., et al., Blood-based NfL: A biomarker for differential diagnosis of parkinsonian disorder. Neurology, 2017.

26. Green, H., et al., Alterations of p11 in brain tissue and peripheral blood leukocytes in Parkinson's disease. Proc Natl Acad Sci USA, 2017.

27. Chatterjee, P. and D. Roy, Comparative analysis of RNA-Seq data from brain and blood samples of Parkinson's disease. Biochem Biophys Res Commun, 2017.

28. O'Bryant, S. E., et al., A serum protein-based algorithm for the detection of Alzheimer disease. Archives of Neurology, 2010. 67(9): p. 1077-1081.

29. O'Bryant, S. E., et al., A blood-based algorithm for the detection of Alzheimer's disease. Dementia and Geriatric Cognitive Disorders, 2011. 32(1): p. 55-62.

30. O'Bryant, S. E., et al., A Blood-Based Screening Tool for Alzheimer's Disease That Spans Serum and Plasma: Findings from TARC and ADNI. PLoS ONE, 2011. 6(12): p. e28092.

31. O'Bryant S E, E. M., Johnson L A, Hall J A, Villarreal A E, Britton G B, Quiceno M, Cullum C M, Graff-Radford N R, A Blood Screening Test for Alzheimer's Disease. Alzheimer's & Dementia: Diagnosis, Assessment & Disease Monitoring, 2016. 3: p. 83-90.

32. O'Bryant, S. E., et al., Validation of a serum screen for alzheimer's disease across assay platforms, species, and tissues. Journal of Alzheimer's Disease, 2014. 42(4): p. 1325-1335.

33. Cho, H. H., et al., Selective translational control of the Alzheimer amyloid precursor protein transcript by iron regulatory protein-1. J Biol Chem, 2010. 285(41): p. 31217-32.

34. Ding, H., et al., Association of SNCA with Parkinson: replication in the Harvard NeuroDiscovery Center Biomarker Study. Mov Disord, 2011. 26(12): p. 2283-6.

35. Hakimi, M., et al., Parkinson's disease-linked LRRK2 is expressed in circulating and tissue immune cells and upregulated following recognition of microbial structures. J Neural Transm, 2011. 118(5): p. 795-808.

36. Hu, Y., et al., Transcriptional modulator H2A histone family, member Y (H2AFY) marks Huntington disease activity in man and mouse. Proc Natl Acad Sci USA, 2011. 108(41): p. 17141-6.

37. Scherzer, C. R., et al., Molecular markers of early Parkinson's disease based on gene expression in blood. Proc Natl Acad Sci USA 2007. 104: p. 955-960.

38. Scherzer, C. R., et al., GATA transcription factors directly regulate the Parkinson's disease-linked gene alphα-synuclein. Proc Natl Acad Sci USA, 2008. 105(31): p. 10907-12.

39. van Blitterswijk, M., et al., Anti-superoxide dismutase antibodies are associated with survival in patients with sporadic amyotrophic lateral sclerosis. Amyotroph Lateral Scler, 2011. 12(6): p. 430-8.

40. Locascio, J. J., et al., Association between alphα-synuclein blood transcripts and early, neuroimaging-supported Parkinson's disease. Brain, 2015. 138(Pt 9): p. 2659-71.

41. Villarreal A E, O. B. S., Edwards M, Grajales S & Britton G B for the Panama Aging Research Initiative, Serum-based protein profiles of Alzheimer's disease and mild cognitive impairment in elderly Hispanics. Neurodegener Dis Manag, 2016, in press.

42. Edwards, M., et al., Molecular markers of amnestic mild cognitive impairment among Mexican Americans. J Alzheimers Dis, 2016. 49(1): p. 221-8.

43. O'Bryant, S. E., et al., Biomarkers of Alzheimer's disease among Mexican Americans. Journal of Alzheimer's Disease, 2013. 34(4): p. 841-849.

44. R_Development_Core_Team. R: A language and environment for statistical computing. 2009; Available from: http://www.R-project.org.

45. Kim, S. W., et al., Interactions between pro-inflammatory cytokines and statins on depression in patients with acute coronary syndrome. Prog Neuropsychopharmacol Biol Psychiatry, 2018. 80(Pt C): p. 250-254.

46. Landers, M. R., et al., A High-Intensity Exercise Boot Camp for Persons With Parkinson Disease: A Phase II, Pragmatic, Randomized Clinical Trial of Feasibility, Safety, Signal of Efficacy, and Disease Mechanisms. J Neurol Phys Ther, 2019. 43(1): p. 12-25.

47. Al-Jarrah, M. D. and N. S. Erekat, Treadmill exercise training could attenuate the upregulation of Interleukin-1beta and tumor necrosis factor alpha in the skeletal muscle of mouse model of chronic/progressive Parkinson disease. NeuroRehabilitation, 2018.

48. Mollenhauer, B., et al., Serum heart-type fatty acid-binding protein and cerebrospinal fluid tau: marker candidates for dementia with Lewy bodies. Neurodegener Dis, 2007. 4(5): p. 366-75.

49. O'Bryant S E, G. V., Henriksen K, Edwards M, Jeromin A, Lista S, Bazenet C, Soares H, Lovestone S, Hampel H, Montine T, Blennow K, Foroud T, Carrillo M, Graff-Radford N, Laske C, Breteler M, Shaw L, Trojanowski J Q, Schupf N, Rissman R, Fagan A M, Oberoi P, Umek R, Weiner M W, Grammas P, Posner H & Martins R, Guidelines for the standardization for preanalytic variables for blood-based biomarker studies in Alzheimer's disease. Alzheimer's & Dementia, 2015. 11: p. 549-60.

1. A method for detecting biomarkers within a primary care setting comprising:
measuring a level of four or more biomarkers selected from IL1, IL7, TNFα, IL5, IL6, CRP, IL10, TNC, ICAM1, FVII, I309, TNFR1, A2M, TARC, adiponectin, MIP1, eotaxin3, sVCAM1, TPO, FABP, IL18, B2M, SAA, PPY, DJ1, and α-synuclein in a sample separated from a human subject in the primary care setting with neurological disease with a nucleic acid, an immunoassay or an enzymatic activity assay.

2. The method of claim 1, wherein the neurological disease is selected from the group consisting of Alzheimer's Disease, Parkinson's Disease, Down's syndrome, Frontotemporal dementia, Dementia with Lewy Bodies.

3. The method of claim 1, wherein the neurological disease is selected from the group consisting of Alzheimer's Disease or Parkinson's Disease.

4. The method of claim 1, wherein the neurological disease is selected from the group consisting of Alzheimer's Disease or Dementia with Lewy Bodies.

5. The method of claim 1, wherein the neurological disease is selected from the group consisting of Parkinson's Disease or Dementia with Lewy Bodies.

6. The method of claim 1, wherein the neurological disease is selected from the group consisting of Alzheimer's Disease, Parkinson's Disease, or Dementia with Lewy Bodies.

7. The method of claim 1, wherein the method detects 5, 6, 7, 8, 9, 10, 11, 12, or 13 biomarkers of neurological diseases.

8. The method of claim 1, wherein the sample is serum or plasma.

9. The method of claim 1, further comprising the step of obtaining the following parameters: patient age, and a neurocognitive screening tests, wherein the combination of two or more serum-based markers, age and the neurocognitive screening tests) are at least 90% accurate in a primary care setting for the determination of Alzheimer's disease when compared to a control subject that does not have a neurological disease or disorder.

10. The method of claim 9, wherein a profile comprises age, sVCAM1, IL5, B2M, IL6, IL1, adiponexin, Eotaxin, MIP1 and IL10.

11. The method of claim 9, wherein a profile comprises NFL, PPY, FABP3, IL18, IL7, TARC, TPO, α-syn, Eotaxin3 and IL5, and further comprises Ab40, Ab42, tau, alpha-syn, and NfL.

12. The method of claim 1, further comprising the step of determining one or more of the following parameters: sleep disturbance (yes/no), visual hallucinations (yes/no), psychiatric/personality changes (yes/no), age, neurocognitive screening, and two or more serum-based markers for the accurate detection and discrimination between neurodegenerative diseases.

13. The method of claim 1, wherein the level of expression identified by nucleic acid, an immunoassay or an enzymatic activity assay is selected from fluorescence detection, chemiluminescence detection, electrochemiluminescence detection and patterned arrays, reverse transcriptase-polymerase chain reaction, antibody binding, fluorescence activated sorting, detectable bead sorting, antibody arrays, microarrays, enzymatic arrays, receptor binding arrays, allele specific primer extension, target specific primer extension, solid-phase binding arrays, liquid phase binding arrays, fluorescent resonance transfer, or radioactive labeling.

14. The method of claim 1, wherein the method is used to screen for at least one of mild AD (CDR global score <=1.0) with an overall accuracy of 94, 95, 96, 97, 98, 99 or 100% (sensitivity (SN), specificity (SP) of (SN=0.94, SP=0.83)), or very early AD (CDR global score=0.5), with an overall accuracy of 91, 92, 93, 94, 95, 96, 97, 98, 99, or 100% (SN=0.97, SP=0.72).

15. The method of claim 1, wherein the method is used to screen in the primary setting uses a higher specificity than sensitivity, wherein the specificity is in the range of 0.97 to 1.0, and the sensitivity is in the range of 0.80 to 1.0.

16. A method for detecting biomarkers in a human patient with neurological disease, the method comprising:
detecting a level of four or more proteins selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and α-synuclein by separating the proteins in a sample separated from a human subject in the primary care setting with neurological disease contained in the sample and a molecular marker by electrophoresis;
contacting the separated proteins with four or more antibodies that each specifically bind to four or more proteins selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and α-synuclein, and thereafter with a secondary antibody; and then
detecting the presence of IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and α-synuclein according to the molecular weight marker.

17. The method of claim 16, wherein the secondary antibody comprises a fluorescence label, chemiluminescence label, a electrochemiluminescence label, the separation is on a patterned arrayan antibody arrays, a fluorescent resonance transfer label, or a radioactive label.

18. The method of claim 16, wherein the neurological disease is selected from the group consisting of Alzheimer's Disease, Parkinson's Disease, Down's syndrome, Frontotemporal dementia, Dementia with Lewy Bodies.

19. The method of claim 16, wherein the neurological disease is selected from the group consisting of Alzheimer's Disease or Parkinson's Disease.

20. The method of claim 16, wherein the neurological disease is selected from the group consisting of Alzheimer's Disease or Dementia with Lewy Bodies.

21. The method of claim 16, wherein the neurological disease is selected from the group consisting of Parkinson's Disease or Dementia with Lewy Bodies.

22. The method of claim 16, wherein the neurological disease is selected from the group consisting of Alzheimer's Disease, Parkinson's Disease, or Dementia with Lewy Bodies.

23. The method of claim 16, wherein the method detects 5, 6, 7, 8, 9, 10, 11, 12, or 13 biomarkers of neurological diseases.

24. The method of claim 16, wherein the sample is serum or plasma.

25. The method of claim 16, further comprising the step of obtaining the following parameters: patient age, and a neurocognitive screening tests, wherein the combination of two or more bioserum-based markers, age and the neurocognitive screening tests) are at least 90% accurate in a primary care setting for the determination of Alzheimer's disease when compared to a control subject that does not have a neurological disease or disorder.

26. The method of claim 25, wherein a profile comprises age, sVCAM1, IL5, B2M, IL6, adiponexin, Eotaxin, MIP1 and IL10.

27. The method of claim 25, wherein a profile comprises NFL, PPY, FABP3, IL18, IL7, TARC, TPO, α-syn, Eotaxin3 and IL5, and further comprises Ab40, Ab42, tau, alpha-syn, and NfL.

28. The method of claim 16, further comprising the step of determining one or more of the following parameters: sleep disturbance (yes/no), visual hallucinations (yes/no), psychiatric/personality changes (yes/no), age, neurocognitive screening, and two or more serum-based biomarkers for the accurate detection and discrimination between neurodegenerative diseases.

29. The method of claim 16, wherein the method is used to screen in the primary setting uses a higher specificity than sensitivity, wherein the specificity is in the range of 0.97 to 1.0, and the sensitivity is in the range of 0.80 to 1.0.

30. A method of selecting subjects for a clinical trial to evaluate a candidate drug believed to be useful in treating neurological diseases, the method comprising:
measuring a level of four or more biomarkers selected from IL7, TNFα, IL5, IL6, CRP, IL10, TNC, ICAM1, FVII, I309, TNFR1, A2M, TARC, eotaxin3, VCAM1, TPO, FABP, IL18, B2M, SAA, PPY, DJ1, and α-synuclein in a sample separated from a human subject in the primary care setting with neurological disease with a nucleic acid, an immunoassay or an enzymatic activity assay; and
determining if the subject should participate in the clinical trial based on the results of the identification of the neurodegenerative disease profile of the subject obtained from the step (a), wherein the subject is only selected if the neurodegenerative disease profile if the candidate drug is likely to be useful in treating the neurological disease.

31. A method of evaluating the effect of a treatment for a neurological disease, the method comprising:
treating a patient for a neurological disease;
measuring a level of four or more biomarkers selected from IL7, TNFα, IL5, IL6, CRP, TNC, ICAM1, FVII, I309, TNFR1, A2M, TARC, eotaxin3, VCAM1, TPO, FABP, IL18, B2M, SAA, PPY, DJ1, and α-synuclein in a sample separated from a human subject in the primary care setting with neurological disease with a nucleic acid, an immunoassay or an enzymatic activity assay; and
determining if the treatment reduces the expression of the one or more biomarkers that is statistically significant as compared to any reduction occurring in the second subset of patients that have not been treated or from a prior sample obtained from the patient, wherein a statistically significant reduction indicates that the treatment is useful in treating the neurological disease.
